(19) United States
(12) Patent Application Publication
(10) Pub. No.: US 2007/0105192 A1
(43) Pub. Date: May 10, 2007

(54) Cyclodextrin affinity purification

(76) Inventors: Joseph John Villafranca, New Hope, PA (US); David James Hakes, Willow Grove, PA (US); Karl F. Johnson, Hatboro, PA (US); Walter Scott Willett, Doylestown, PA (US); Chester A. Meyers, Medford, NJ (US)

Correspondence Address: MORGAN, LEWIS & BOCKIUS LLP (SF), 2 PALO ALTO SQUARE, 3000 El Camino Real, Suite 700, PALO ALTO, CA, 94306, US

(21) Appl. No.: 10/555,123

(22) PCT Filed: May 5, 2004
(86) PCT No.: PCT/US04/13841

§ 371 Date: Aug. 7, 2006

(30) Foreign Application Priority Data
May 5, 2003 (US) 60468374

(51) Int. Cl. C12P 21/06; C12P 19/62; C12P 19/28; C12P 19/04; C12N 9/10

(52) U.S. Cl. 435/69.1; 435/85; 435/101; 435/193; 435/76

(57) ABSTRACT

A method of immobilizing a molecular species that include a starch-binding domain is provided. There also is provided a material upon which the molecular species is immobilized, and a material that is capable of immobilizing the species The method includes binding the species to a solid support, e.g., membranes, chromatographic supports and the like. The immobilized species is optionally purified by the method of the invention. Alternatively, the immobilized species is use in another method, such as in a synthesis as a synthetic reagent, or to purify another species that has an affinity for the immobilized species. Exemplary immobilized molecular species include bioactive agents, and biomolecules.

### BACKGROUND OF THE INVENTION

Methods for isolating and/or detecting recombinant proteins of interest are useful in a number of applications. For instance, sensitive detection of transgene products in genetically engineered animals is important in determining the tissues in which transgene expression occurs. The proteins can be detected using a binding ligand (e.g., an antibody) that specifically recognizes the desired protein. In most cases, this procedure requires raising antibodies that are specifically immunoreactive with the desired protein. To avoid this requirement, various tags which can be fused to the protein of interest have been developed. For instance, the tags may include a unique epitope for which antibodies are readily available. Other methods include use of tags which incorporate metal-chelating amino acids.

Recombinant fusion proteins with a molecular “purification tag” at one end are known in the art. The purification, tag facilitates purification of the protein. Such tags can also be used for immobilization of a protein of interest during reactions, assays or detection processes. Suitable tags include “epitope tags,” which are a peptide sequence that is specifically recognized by a recognition moiety. Epitope tags are generally incorporated into fusion proteins to enable the use of a readily available recognition moiety to unambiguously detect or isolate the fusion protein. A “FLAG tag” is a commonly used epitope tag, specifically recognized by a monoclonal anti-FLAG recognition moiety, consisting of the sequence AspTyrLysAspAspAspAspLys (SEQ ID NO. 1) or a substantially identical variant thereof. Other suitable tags are known to those of skill in the art, and include, for example, an affinity tag such as a hexahistidine peptide, which will bind to metal ions such as nickel or cobalt ions. Purification tags also include maltose binding domains and starch binding domains. Purification of maltose binding domain proteins is know to those of skill in the art. Starch binding domains are described in WO 99/15636, herein incorporated by reference.

The affinity of cellulases for cellulose have been used for their purification (Boyer et al., Biotechnol. Bioeng. (1987) 29:176-179; Halliwell et al., Bio-chem. Chem J. (1978) 169:713-735; Martyanov et al., Biokhi-miya (1984) 19:405-104; Nummi et al., Anal Biochem. (1981) 116:137-141; van Tilbeurgh et al., FEBS Letters (1986) 204:223-227). Several cellulase genes from Cellulomonas fimi have been cloned into Escherichia coli (Whittle et Binding to Avicel (microcrystalline cellulose) has been used for purification of both native (Gilkes et al., J. Biol. Chem. (1984) 259:10455-10459) and recombinant enzymes (Owolabi et al., Appl. Environ. Microbiol. (1988) 54:518-523). A bifunctional hybrid protein which binds maltose has been described Bedouelle et al., Eur. J. Biochem. (1988) 171:541-549.

Heparin affinity chromatography using heparin-Sepharose.RTM. was first used to purify a tumor-derived angiogenic endothelial mitogen in 1984 (Shing et al. (1984) Science 223: 1296-1298). Heparin affinity chromatography has since been widely used for the purification of fibroblast growth factors from a large variety of tissue sources (for reviews see Folkman and Klagsbrun (1987) Science 235: 442-447; Baird et al. (1986) Recent Prog. Horm. Res. 43: 143-205; Gospodarowicz et al. (1986) Mol. Cell. Endocrinol. 46: 187-204; and Lobb et al., (1986) Anal. Biochem. 154: 1-14).

Cyclodextrin glucanotranisferase was purified on affinity sorbents that include α- and β-cyclodextrins. No mention is made that the immobilized enzyme would be of use as a synthetic reagent or for performing analyses or assays.

Compositions that are immobilized to supports by the interaction between a saccharide binding domain and a moiety that is recognized by the saccharide-binding domain would be of use as supported reagents for synthesis and as substrates and reagents for performing assays and analysis. The present invention provides such compositions and methods of using them.

### BRIEF SUMMARY OF THE INVENTION

Synthesis using immobilized reagents or substrates offers numerous advantages over conventional solution phase chemistries. The benefits of solid-phase synthetic methodologies are no where better illustrated than in the wide spread acceptance of and success enjoyed by solid phase peptide and nucleic acid synthetic techniques. Despite the utility of solid phase methodologies, their application to the synthesis of saccharides is not as widely accepted as those methods by which peptides and nucleic acids are prepared.

One of the most promising methods for preparing saccharides relies on enzymes that naturally transfer a glycosyl residue to a saccharidyl or peptidyl acceptor. The chemical immobilization of such an enzyme on a solid support involves the risk that one or more site essential to the activity of the enzyme will be the locus at which the enzyme becomes Thus, methods of immobilizing an enzyme on a solid support through a group that is not implicated in the enzymes reactivity are highly desirable.

The present invention provides compositions that include a starch-binding domain (SBD) within their structure and methods for using the compounds. Exemplary compositions are enzymes, such as those of use in assembling saccharides, e.g., glycosyltransferases. The invention also provides a solid support on which a recognition moiety, e.g., a saccharide is bound. The saccharide is recognized by the SBD. When the SBD is conjugated to a species of interest, the species can be immobilized on the solid support through the interaction between the SBD and the support-bound saccharide. The combination of the SBD-labeled species and the solid support is useful in methods for synthesis using immobilized reagents, and removal of reagents from a reaction media. The invention also provides a solid support with a recognition moiety for a SBD and a method for analyzing a sample for the presence of a species that binds to the immobilized recognition moiety.

Thus, in a first aspect, the present invention provides a method for immobilizing a species onto a solid support. The species includes a SBD and the solid support includes a saccharide that interacts with the SBD to immobilize the species on the solid support. In an exemplary embodiment, the species immobilized according to the method of the invention is a reagent, e.g., an enzyme for effecting a chemical transformation on a substrate. The enzyme, substrate or both are immobilized on the support at a selected step of the reaction pathway. For example, there is provided a method of performing an enzymatic transformation on a substrate that includes a starch-binding domain. The SBC is used to immobilize the substrate (or the reaction product) on a solid support. Alternatively, the enzyme includes a starch-binding domain and it is immobilized on the solid support before, during or following the transformation.

In another aspect, the invention provides a method for performing a chemical transformation on a substrate. The method includes (a) contacting the substrate with a reagent under conditions suitable to perform the transformation, wherein the reagent includes a starch-binding domain; and (b) immobilizing the reagent on a support that includes a cyclodextrin by binding the starch-binding domain to the cyclodextrin. An exemplary reagent is an enzyme. The method includes, (a) contacting a glycosyl donor moiety and an acceptor for the glycosyl donor moiety with a glycosyltransferase having a starch-binding domain under conditions suitable to transfer the glycosyl donor moiety to the substrate; and (b) immobilizing the glycosyltransferase having a starch-binding domain on a solid support. The solid support has attached thereto a cyclodextrin that interacts with the starch-binding domain, thereby immobilizing the glycosyltransferase on the cyclodextrin. Step (b) can be performed either before, during or after glycosylation.

The invention also provides a solid support that has a saccharide bound thereto, which is recognized by the SBD. In an exemplary embodiment, the solid support has a cyclodextrin moiety bound thereto. In yet another exemplary embodiment, an enzyme is bound to the solid support. The enzyme includes a starch-binding domain, and the starch-binding domain interacts with the cyclodextrin immobilizing said glycosyltransferase on said solid support.

In another aspect, the invention provides a material that includes a solid support having a cyclodextrin moiety bound thereto; and a species comprising a starch-binding domain bound thereto. The starch-binding domain interacts with the cyclodextrin, thereby immobilizing the species on the solid support.

Other aspects, objects and advantages of the present invention are apparent from the detailed description that follows.

### BRIEF DESCRIPTION OF THE DRAWINGS

FIG. 1 is a cartoon showing the process of preparing a glycosyltransferase fusion protein that includes a SBD; the use of the fusion protein to alter the glycosylation pattern on a therapeutic peptide and the removal of the fusion protein from the reaction mixture using the affinity of the SBD for a solid support having a saccharide bound thereto.

FIG. 2 is a profile of the elution conditions for immobilizing and removing a fusion protein from the saccharide-bearing support.

FIG. 3 is a chromatogram of the affinity chromatography of the harvest of fusion protein; and a gel showing the presence of the fusion in selected fractions. [symbol] broth of fusion protein; and a gel showing the presence of the fusion in selected fractions.

FIG. 5 is a chromatogram of the affinity chromatography of the SPFF pool; and a gel showing the presence of the fusion in selected fractions.

FIG. 6 is a Western Blot using an anti-ST3GalIII antibody blotted against the SBD/ST3GalIII fusion protein expressed in the vector/JM109.

FIG. 7 is the nucleic acid sequence glaA (glucoamylase gene) from A. awamori including 5′ flanking sequences (SEQ ID NO. 2): using techniques known to those skilled in the art one can either express the whole gene in a system that will splice out the introns in this sequence or use PCR to generate a construct containing only the coding sequence. Initiating methionine of signal peptide is at nuc 260-262.

FIG. 8 is the nucleotide sequence of SBD domain from A. awamori (SEQ ID NO. 3).

FIG. 9 is the amino acid sequence of G1 form of glucoamylase including signal peptide (SEQ ID NO. 4).

FIG. 10 is the amino acid sequence of the SBD from glucoamylase (SEQ ID NO. 5).

### DETAILED DESCRIPTION OF THE INVENTION

Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly syntheses and chemical analyses.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques. A “recombinant protein” is one produced by a recombinant cell.

The term “swapping” refers to the recombinant manipulation of nucleic acid sequence or amino acid sequence to construct the fusion proteins of the invention as described herein, and is not limited to the exchange or replacement of nucleic acid sequences or amino acid sequences. For example, nucleic acid sequence or amino acid sequence can be extended, shortened or modified to construct the fusion proteins of the invention. Also for example, a nucleic acid sequence or amino acid sequence of a first glycosyltransferase can be modified to contain sequences that are substantially identical to the nucleic acid sequence or amino acid sequence, respectively, of a second glycosyltransferase and, thereby, a “fusion protein” is constructed.

A “fusion protein” refers to a protein comprising amino acid sequences that are in addition to, in place of, less than, and/or different from the amino acid sequences encoding the original or native fall-length protein or subsequences thereof.

Components of fusion proteins include “accessory enzymes” and/or “purification tags.” An “accessory enzyme” as referred to herein, is an enzyme that is involved in catalyzing a reaction that, for example, forms a substrate for a glycosyltransferase. An accessory enzyme can, for example, catalyze the formation of a nucleotide sugar that is used as a donor moiety by a glycosyltransferase. An accessory enzyme can also be one that is used in the generation of a nucleotide triphosphate required for formation of a nucleotide sugar, or in the generation of the sugar which is incorporated into the nucleotide sugar. The term “functional domain” with reference to glycosyltransferases, refers to a domain of the glycosyltransferase that confers or modulates an activity of the enzyme, e.g. acceptor apparatus, anchoring to a cell membrane, or other biological or biochemical activity. Examples of functional domains of glycosyltransferases include, but are not limited to, the catalytic domain, stem region, and signal-anchor domain.

The terms “expression level” or “level of expression” with reference to a protein refers to the amount of a protein produced by a cell. In a preferred embodiment, the protein is a recombinant glycosyltransferase fusion protein having a “high” level of expression, which refers to an optimal amount of protein useful in the methods of the present invention. The amount of protein produced by a cell can be measured by the assays and activity units described herein or known to one skilled in the art. One skilled in the art would know how to measure and describe the amount of protein produced by a cell using a variety of assays and units, respectively. Thus, the quantitation and quantitative description of the level of expression of a protein, e.g., a glycosyltransferase, is not limited to the assays used to measure the activity or the units used to describe the activity, respectively. The amount of protein produced by a cell can be determined by standard known assays, for example, the protein assay by Bradford (1976), the bicinchoninic acid protein assay kit from Pierce (Rockford, Ill.), or as described in U.S. Pat. No. 5,641,668.

The term “enzymatic activity” refers to an activity of an enzyme and may be measured by the assays and units described herein or known to one skilled in the art. Examples of an activity of a glycosyltransferase include, but are not limited to, those associated with the functional domains of the enzyme, e.g., acceptor substrate specificity, catalytic activity, binding affinity, localization within the Golgi apparatus, anchoring to a cell membrane, or other biological or biochemical activity. In a preferred embodiment, the enzyme has “high” enzymatic activity which refers to an optimal level of enzymatic activity measured by the assays and units described herein or known to one skilled in the art (see, e.g., U.S. Pat. No. 5,641,668). One skilled in the art knows how to measure and describe an enzyme activity using a variety of assays and units, respectively. Thus, the quantitation and quantitative description of an enzymatic activity of a glycosyltransferase is not limited to the assays used to measure the activity or the units used to describe the activity, respectively. Examples of glycosyltransferases having high specific activity include, but are not limited to, the recombinant glycosyltransferase fusion proteins of the invention having a catalytic activity of at least about 0.01 unit/mL, more preferably from 0.05 to 5 units/mL, and most preferably from 5 to 100 units/mL. Other examples of glycosyltransferases having high proteins of the present invention that fucosylate at least 60% of the targeted glycoprotein-linked fucosyltransferase acceptor sites present in a population of glycoproteins in the fucosylation reaction mixture.

The term “specific activity” as used herein refers to the catalytic activity of an enzyme, e.g., a recombinant glycosyltransferase fusion protein of the present invention, and may be expressed in activity units. As used herein, one activity unit catalyzes the formation of 1 μmol of product per minute at a given temperature (e.g., at 37° C.) and pH value (e.g., at pH 7.5). Thus, 10 units of an enzyme is an amount of enzyme sufficient to catalyze the conversion of 10 μmol of substrate into 10 μmol of product in one minute at a selected temperature, e.g., 37° C. and a selected pH value, e.g., 7.5.

A “stem region” with reference to glycosyltransferases refers to a protein domain, or a subsequence thereof, which in the native glycosyltransferases is located adjacent to the trans-membrane domain, and known to function as a retention signal to maintain the glycosyltransferase in the Golgi apparatus, and as a site of proteolytic cleavage. An exemplary stem region is the stem region of fucosyltransferase VI, amino acid residues 40-54.

A “catalytic domain” refers to a protein domain, or a subsequence thereof, that catalyzes an enzymatic reaction performed by the enzyme. For example, a catalytic domain of a sialyltransferase will include a subsequence of the sialyltransferase sufficient to transfer a sialic acid residue from a donor to an acceptor saccharide. A catalytic domain can include an entire enzyme, a subsequence thereof, or can include additional amino acid sequences that are not attached to the enzyme, or a subsequence thereof, as found in nature. An exemplary catalytic region is the catalytic domain of fucosyltransferase VII, amino acid residues 39-342.

A “subsequence” refers to a sequence of nucleic acids or amino acids that are a subset or a part of a longer sequence of nucleic acids or amino acids (e.g., protein) respectively.

The term “nucleic acid” refers to a deoxyribonucleotide or ribonucleotide polymer in either single-or double-stranded form, and unless otherwise limited, encompasses known analogues of natural nucleotides that hybridize to nucleic acids in a manner similar to [symbol] sequence includes the complementary sequence thereof.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

A “heterologous sequence” or a “heterologous nucleic acid”, as used herein, is one that originates from a source foreign to the particular host cell, or, if from the same source, is modified from its original form. Thus, a heterologous glycoprotein gene in a eukaryotic host cell includes a glycoprotein-encoding gene that is endogenous to the particular host cell that has been modified. Modification of the heterologous sequence may occur, e.g., by treating the DNA with a restriction enzyme to generate a DNA fragment that is capable of being operably linked to the promoter. Techniques such as site-directed mutagenesis are also useful for modifying a heterologous sequence.

The term “isolated” refers to material that is substantially or essentially free from components other than the desired product. For a saccharide, protein, or nucleic acid of the invention, the term “isolated” refers to material that is substantially or essentially free from components that normally accompany the material as found in its native state. Typically, an isolated saccharide, protein, or nucleic acid of the invention is at least about 80% pure, usually at least about 90%, and preferably at least about 95% pure as measured by band intensity on a silver stained gel or other method for determining purity. Purity or homogeneity can be indicated by a number of means well known in the art. For example, a protein or nucleic acid in a sample can be resolved by polyacrylamide gel electrophoresis, and then the protein or nucleic acid can be visualized by staining. For certain purposes high purification, for example, may be utilized.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

The terms “identical” or percent “identity,” in the context of two or more nucleic acids or protein sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or proteins, refers to two or more sequences or subsequences that have at least greater than about 60% nucleic acid or amino acid sequence identity, 65%, 70%, 75%, 80%, 85%, 90%, preferably 91%, 92%, 93%, 94%, 95%, 96%, 97%, 98% or 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff& Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & [symbol] provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or proteins are substantially identical is that the protein encoded by the first nucleic acid is immunologically cross reactive with the protein encoded by the second nucleic acid, as described below. Thus, a protein is typically substantially identical to a second protein, for example, where the two peptides differ only by conservative substitutions. Another indication that two nucleic acid sequences are substantially identical is that the two molecules hybridize to each other under stringent conditions, as described below.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 15° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na+ion, typically about 0.01 to 1.0 M Na+ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. [symbol] with”, when referring to an recognition moiety refers to a binding reaction which is determinative of the presence of the protein in the presence of a heterogeneous population of proteins and other biologics. Thus, under designated immunoassay conditions, the specified antibodies bind preferentially to a particular protein and do not bind in a significant amount to other proteins present in the sample. Specific binding to a protein under such conditions requires an recognition moiety that is selected for its specificity for a particular protein. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, N.Y., for a description of immunoassay formats and conditions that can be used to determine specific immunoreactivity.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given protein. For instance, the codons CGU, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every polynucleotide sequence described herein, which encodes a protein also describes every possible silent variation, except where otherwise noted. One of skill will recognize that each codon in a nucleic acid (except AUG, which is ordinarily the only codon for methionine, and UGG which is ordinarily the only codon for tryptophan) can be modified to yield a functionally identical molecule by standard techniques. Accordingly, each “silent variation” of a nucleic acid, which encodes a protein is implicit in each described sequence.

Furthermore, one of skill will recognize that individual substitutions, deletions or additions which alter, add, or delete a single amino acid or a small percentage of amino acids (typically less than 5%, more typically less than 1%) in an encoded sequence are “conservatively modified variations” where the alterations result in the substitution of an functionally similar amino acids are well known in the art.

One of skill will appreciate that many conservative variations of the fusion proteins and nucleic acid which encode the fusion proteins yield essentially identical products. For example, due to the degeneracy of the genetic code, “silent substitutions” (ie., substitutions of a nucleic acid sequence which do not result in an alteration in an encoded protein) are an implied feature of every nucleic acid sequence which encodes an amino acid. As described herein, sequences are preferably optimized for expression in a particular host cell used to produce the chimeric endonucleases (e.g., yeast, human, and the like). Similarly, “conservative amino acid substitutions,” in one or a few amino acids in an amino acid sequence are substituted with different amino acids with highly similar properties (see, the definitions section, supra), are also readily identified as being highly similar to a particular amino acid sequence, or to a particular nucleic acid sequence which encodes an amino acid. Such conservatively substituted variations of any particular sequence are a feature of the present invention. See also, Creighton (1984) Proteins, W.H. Freeman and Company. In addition, individual substitutions, deletions or additions which alter, add or delete a single amino acid or a small percentage of amino acids in an encoded sequence are also “conservatively modified variations”.

The practice of this invention can involve the construction of recombinant nucleic acids and the expression of genes in transfected host cells. Molecular cloning techniques to achieve these ends are known in the art. A wide variety of cloning and in vitro amplification methods suitable for the construction of recombinant nucleic acids such as expression vectors are well known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1999 Supplement) (Ausubel). Suitable host cells for expression of the recombinant polypeptides are known to those of skill in the art, and include, for example, eukaryotic cells including insect, mammalian and fungal cells (e.g., Aspergillus niger) [symbol] amplification methods, including the polymerase chain reaction (PCR) the ligase chain reaction (LCR), Qβ-replicase amplification and other RNA polymerase mediated techniques are found in Berger, Sambrook, and Ausubel, as well as Mullis et al. (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039.

“Peptide,” “polypeptide” or “protein” refers to a polymer in which the monomers are amino acids and are joined together through amide bonds, alternatively referred to as a peptide bond. The L-optical isomer or the D-optical isomer can be used. Additionally, unnatural amino acids, for example, β-alanine, phenylglycine and homoarginine are also included. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical acid, but function in a manner similar to a naturally occurring amino acid.

“Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides; carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “heteroalkyl,” by itself or in combination with another term, means, unless otherwise stated, a stable straight or branched chain, or cyclic hydrocarbon radical, or heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—₂—NH—CH₃, —CH₂—CH₂—N(CH₃)—CH₃, —CH₂—S—CH₂—CH₃, —CH₂—CH₂,—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene lining groups, no orientation of the linking group is implied by the direction in which the formula of the linking group is written. For example, the formula —C(O)₂R′— represents both —C(O)₂R′— and —R′C(O)₂—.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, l-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. [symbol] aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″,—S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″,R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, CONR′R″, —OC(O)NR′R″, —NR″C(O)R′, —NR′—C(O)NR″R″′, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

The term “recognition moiety” refers to a moiety that recognizes and interacts with a starch-binding domain. The recognition moiety is generally linked to a solid or semi-solid support.

The moiety that recognizes and binds to the starch-binding domain is generally attached to a solid or semi-solid support by a bond formed by reaction of a reactive functional group on the support and a reactive functional group of complementary reactivity on the recognition moiety. Reactive groups and classes of reactions useful in practicing the present invention are generally those that are well known in the art of bioconjugate chemistry. Currently favored classes of reactions available with reactive chelates are those that proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al., Modification of Proteins; Advances in Chemistry Series, Vol. 198, American Chemical Society, Washington, D.C., 1982.

The recombinant glycosyltransferase fusion proteins of the invention are useful for transferring a saccharide from a donor substrate to an acceptor substrate. The addition generally takes place at the non-reducing end of an oligosaccharide or carbohydrate moiety on a biomolecule. Biomolecules as defined here include but are not limited to biologically glycolipids, phospholipids, sphingolipids and gangliosides).

The term “sialic acid” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neurarminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids: Chemistry, Metabolism and Function, R. Schauer, Ed. (Springer-Verlag, New York (1992)). The synthesis and use of sialic acid compounds in a sialylation procedure is disclosed in international application WO 92/16640, published Oct. 1, 1992.

An “acceptor substrate” for a glycosyltransferase is a chemical species, e.g., a saccharide, or peptide, that can act as an acceptor for a particular glycosyltransferase. When the acceptor substrate is contacted with the corresponding glycosyltransferase and sugar donor substrate, and other necessary reaction mixture components, and the reaction mixture is incubated for a sufficient period of time, the glycosyltransferase transfers sugar residues from the sugar donor substrate to the acceptor substrate. The acceptor substrate will often vary for different types of a particular glycosyltransferase. For example, the acceptor substrate for a mammalian galactoside 2-L-fucosyltransferase (α1,2-fucosyltransferase) will generally include a Galβ1,4-GlcNAc-R at a non-reducing terminus of an oligosaccharide; this fucosyltransferase attaches a fucose residue to the Gal via an α1,2 linkage. Terminal Galβ1,4-GlcNAc-R and Galβ1,3-GlcNAc-R and sialylated analogs thereof are acceptor substrates for α1,3 and α1,4-fucosyltransferases, respectively. These enzymes, however, attach the fucose residue to the GlcNAc residue of the acceptor substrate. Accordingly, the term “acceptor substrate” is taken in context with the particular glycosyltransferase of interest for a particular application. Acceptor substrates for additional fucosyltransferases, and for other glycosyltransferases, are described herein. [symbol] activated sugars generally consist of uridine, guanosine, and cytidine monophosphate derivatives of the sugars (UP, GMP and CMP, respectively) or diphosphate derivatives of the sugars (UDP, GDP and CDP, respectively) in which the nucleoside monophosphate or diphosphate serves as a leaving group. For example, a donor substrate for fucosyltransferases is GDP-fucose. Donor substrates for sialyltransferases, for example, are activated sugar nucleotides comprising the desired sialic acid. For instance, in the case of NeuAc, the activated sugar is CMP-NeuAc.

“Solid supports” of use in practicing the present invention include members selected from art-recognized synthetic supports, separation media and the like, e.g., hollow fibers (Amicon Corporation, Danvers, Mass.), beads (Polysciences, Warrington, Pa.), magnetic beads (Robbin Scientific, Mountain View, Calif.), plates, dishes and flasks (Corning Glass Works, Coming, N.Y.), meshes (Becton Dickinson, Mountain View, Calif.), screens and solid fibers (see Edelman et al., U.S. Pat. No. 3,843,324; see also Kuroda et al., U.S. Pat. No. 4,416,777), membranes (Millipore Corp., Bedford, Mass.), and dipsticks.

Introduction

The present invention provides methods of immobilizing a species onto a solid support through a starch binding domain on the species. Also provided are methods for using the immobilized species for synthesis, detection and purification. The immobilizable species includes an amino acid starch-binding domain (SBD) that binds to a saccharide. In an exemplary embodiment, the SBD is conjugated to the immobilizable species. In another exemplary embodiment, the SBD is a sequence that is recombinantly added to the peptide sequence of the immobilizable species. The SBD is optionally removable from the species to which it is bound. For example, a specific or non-specific protease may be used for enzymatic removal of the SBD.

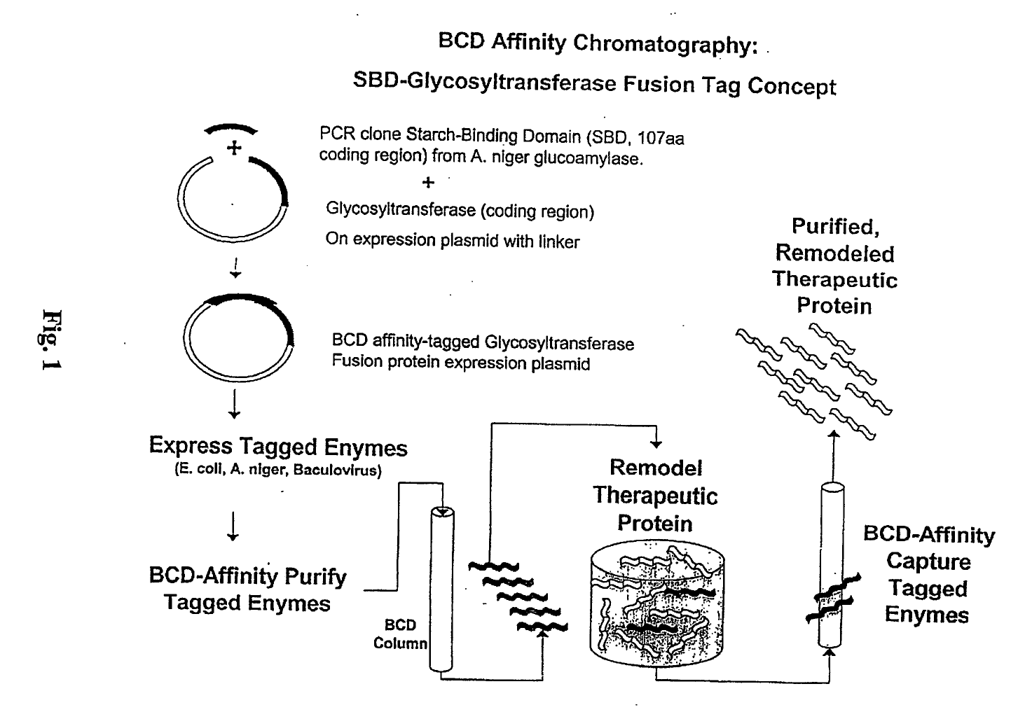

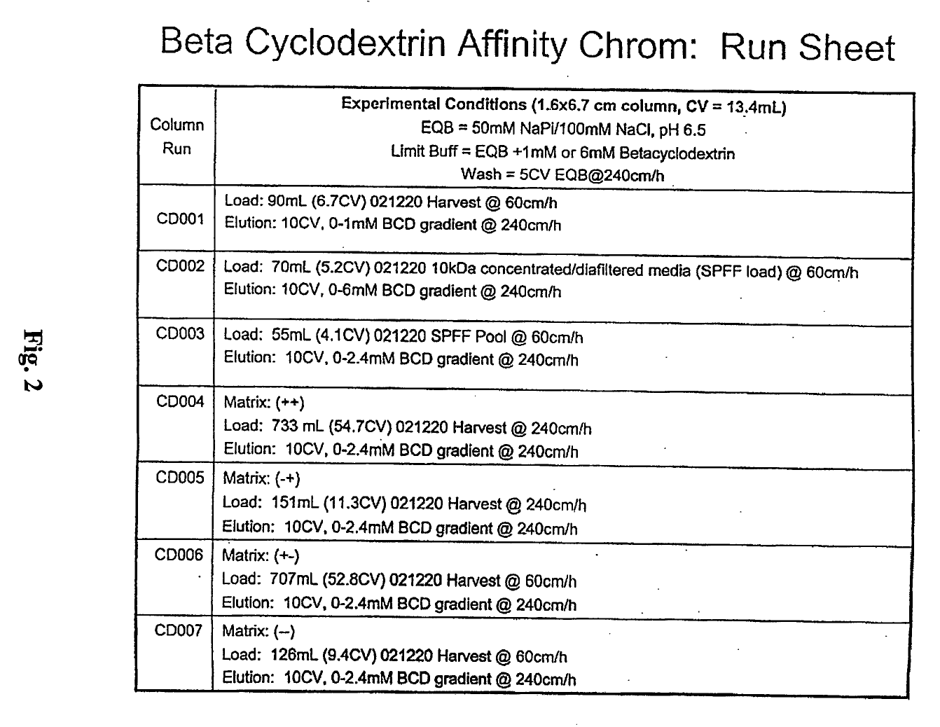

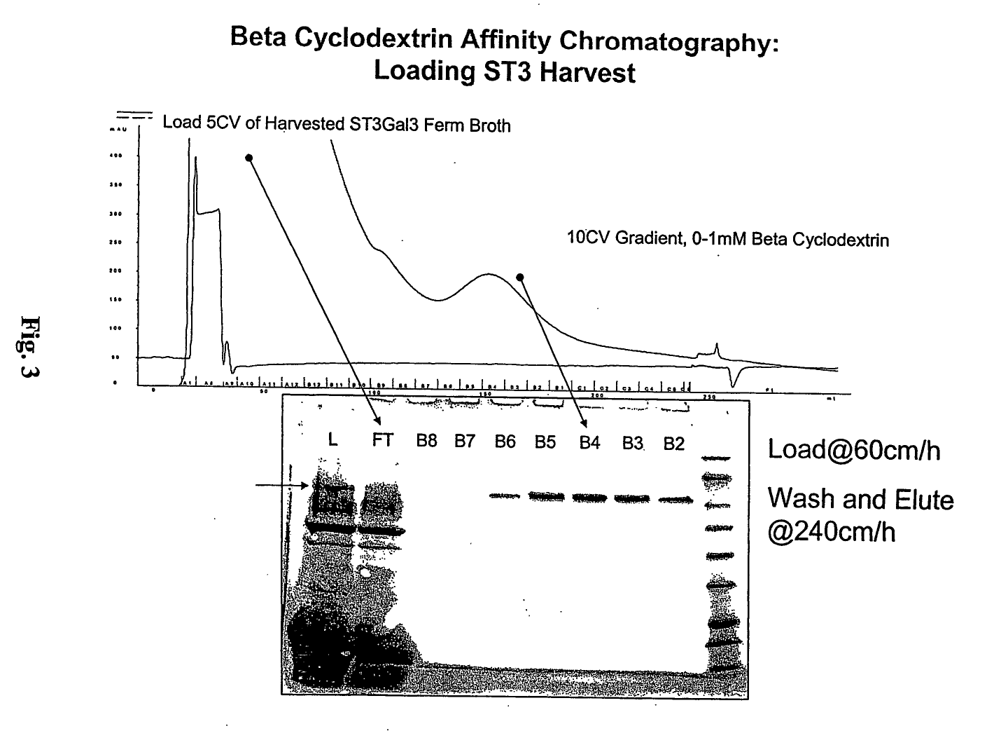

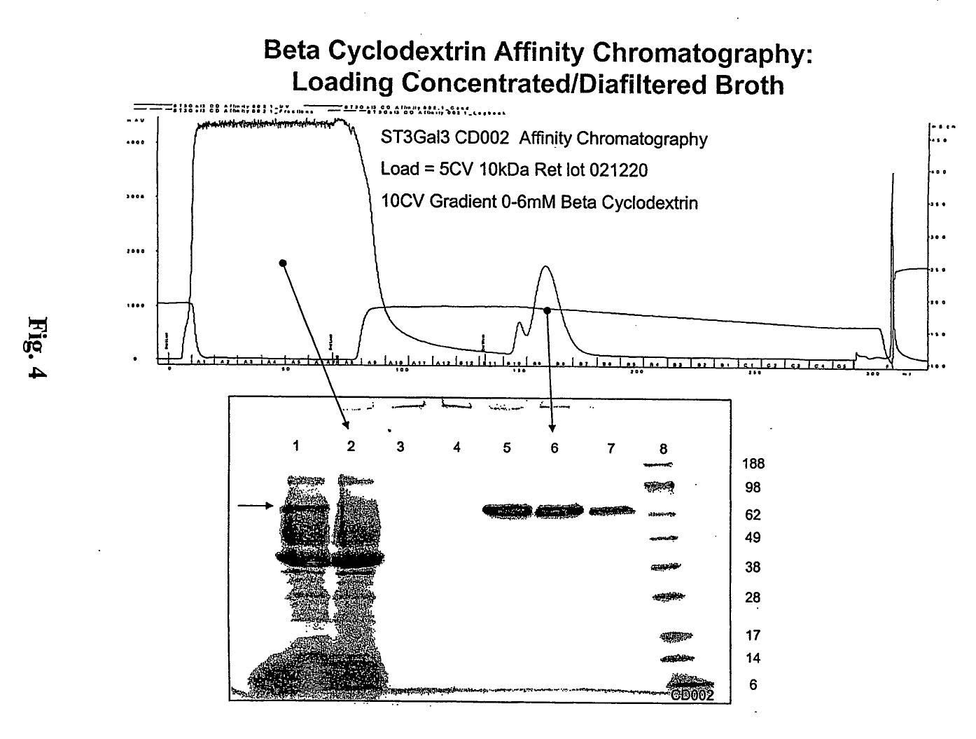

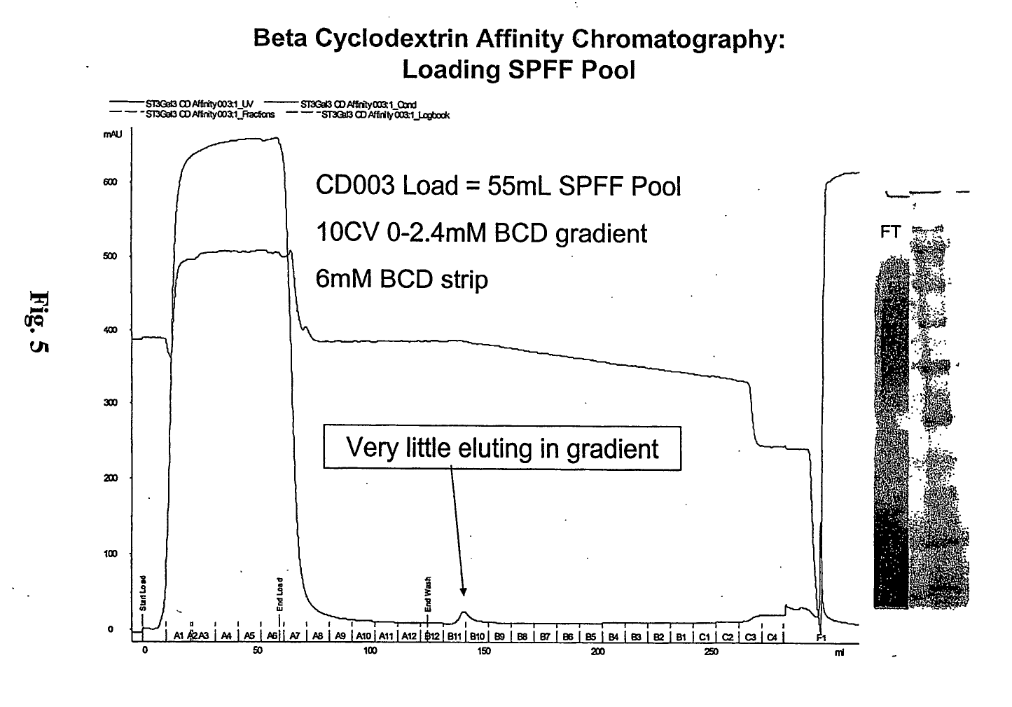

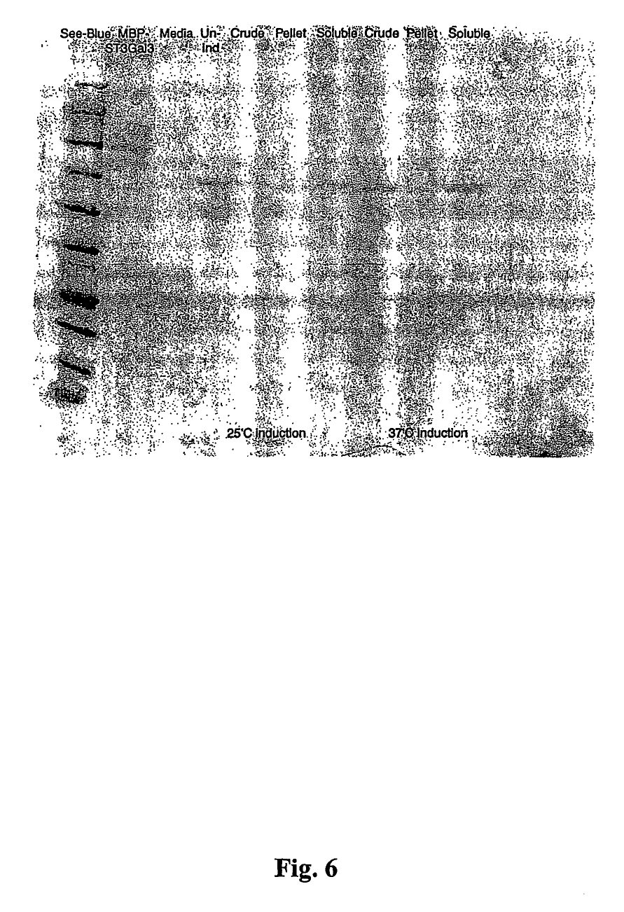

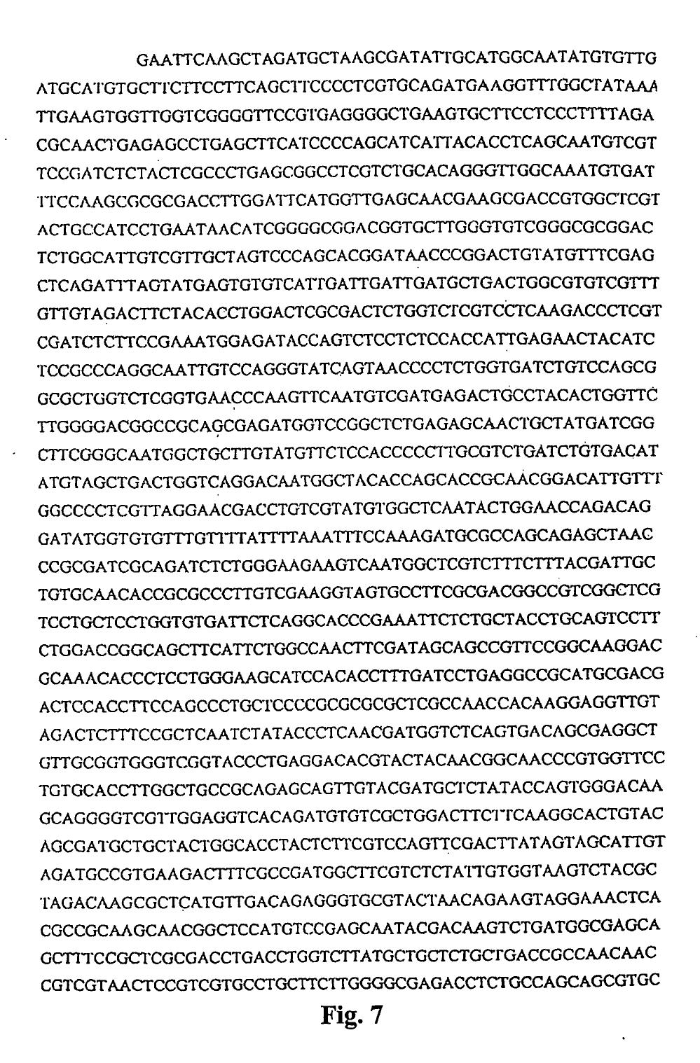

The invention also provides a method for purifying a species that includes a SBD. As shown in FIG. 1, in a method of the invention, a mixture of the SBD-containing species, in this case a glycosyltransferase, is contacted with a saccharide-functionalized support under conditions appropriate for binding the species to the solid support. Impurities that were present in the mixture are washed from the column. Exemplary purification conditions are provided in FIG. 2. Following its purification, the purified species is optionally removed from the support under appropriate conditions and its purity verified if desired (FIG. 3-FIG. [symbol] species can be converted to another species while immobilized, or it can serve as an immobilized reagent suitable for performing a transformation on a substrate. In those embodiments in which the species is removed from the support prior to participating in a reaction, the species can be bound to the support again subsequent to the reaction, thereby allowing the recovery of the species or the purification of the altered substrate.

In an exemplary embodiment, the support-bound SBD-containing species can be removed from the support by contacting the immobilized species with a removal solution capable of eluting the label from the substrate. Alternatively, the SBD may be removed enzymatically by including a protease recognition site within or on one or both flanks of the SBD. Alternatively, there may be placed a chemical cleavage site between the SBD and the species to which it is attached. Exemplary protease cleavage sites include sites for collagenase, thrombin or Factor Xa, which are cleaved specifically by the respective enzymes. In another embodiment the SBD-bearing construct includes a chemical cleavage site that is cleaved under selected conditions, for example, low pH, light, or heat may cleave a bond between the SBD and the species to which it is bound. Alternatively, the entire polysaccharide binding peptide can be degraded by exposure to a relatively non-specific, general protease, such as protease K. Any of these procedures are effective for the removal of the SBD.

In one aspect, the invention provides fusion proteins that include a SBD motif within their amino acid sequence. The fusion proteins provide for a wide variety of applications including purification of the protein of interest, immobilization of the protein of interest, and preparation of solid phase diagnostics, purification of SBD conjugates, and the preparation of coatings, tags and removable dyes. Other applications can include binding a compound of interest to a polysaccharide matrix. The interaction between the SBD and the saccharide-containing support can be used also as a means of purifying compounds, particularly biological compounds.

The compositions can also be used as a means of immobilizing a fusion protein on a polysaccharide support, since the polysaccharide binding domain adsorption to its substrate is strong and specific. The immobilized systems find use, for example, in preparing solid state reagents for diagnostic assays, the reagents including enzymes, antibody fragments, peptide hormones, etc.; drug binding to decrease clearance rate where the support can be either (Avicel) where the drug is a polypeptide such as interleukin 2.

The Starch-Binding Domain

Exemplary SBD moieties of use in the present invention include a structure, e.g., a peptide or saccharide, that is found in a binding domain of a wild type polysaccharide binding protein or a protein designed and engineered to be capable of binding to a polysaccharide. The SBDs found in polysaccharidases provide a useful motif, particularly if the amino acid sequence of the SBD is essentially lacking the hydrolytic enzymatic activity of a polysaccharidase, but retains the substrate binding activity.

The starch-binding domain (SBD) generally includes a peptide sequence that is derived from any glucoamylase gene or any other saccharide-binding protein. Most known SBDs today are found in CGTases, i.e. cyclodextrin glucanotransferases (E.C. 2.4.1.19), and glucoamylases (E.C. 3.2.1.3). See also, Chen et al. (1991), Gene 991: 121-126, describing starch binding domain hybrids. Exemplary SBDs are those that recognize saccharides, such as cellulose, a polysaccharide composed of D-glucopyranose units joined by β-1,4-glycosidic linkages and its esters, e.g. cellulose acetate; xylan, in which the repeating backbone unit is β-1,4-D-xylopyranose; chitin, which resembles cellulose in that it is composed of β-1,4-linked N-acetyl, 2-amino-2-deoxy-β-D-glucopyranose units. Several types of enzymes are involved in the microbial conversion of cellulose and xylan and include endoglucanases (1-4-β-D-glucan glucanohydrolase, EC 3.2.1.4); cellobiohydrolases (1,4-β-D-glucan cellobiohydrolase EC 3.2.1.91); β-glucosidases; xylanases (1,4-β-D-xylan xylanohydrolase, EC 3.2.1.8) and β-xylosidases (1,4-β-D-xylan xylohydrolase, EC 3.2.1.37).

An exemplary SBD is encoded by a glucoamylase gene. The genes encoding the glucoamylase SBDs or fragments thereof can be isolated from any prokaryotic or eukaryotic organism. In one preferred embodiment the glucoamylase gene is from A. awamori. The SBD can be used as a smaller fragment by itself or as part of the larger glucoamylase protein. For example the full length glucoamylase protein or gene can be used (amino acids 1-640 includes signal peptide and represents G1 form) or any of the following forms which include the G2 form of the protein (alternative splicing of transcript omits intron E), the intact G1 or G2 form of the protein containing any nucleotide mutation that disrupts the hydrolytic function of the enzyme (starch degradation amino acids mature peptide 19-488) and any in- [symbol] starch binding domain (mature peptide amino acids 533-640).

The starch-binding domain is incorporated into a fusion protein, as discussed herein, or it is attached chemically to another species, such as a bioactive species or analyte. A presently preferred polysaccharide-binding domain (PBD) is characterized as obtainable from the polysaccharide-binding domain of a polysaccharidase; capable of binding to polysaccharides; and optionally, is essentially lacking in polysaccharidase activity.

The Enzymes

In an exemplary embodiment, the species immobilized by binding of the SBD to the solid support is a polypeptide with glycosyltransferases (e.g., fucosyltransferase) activity. Glycosyltransferases catalyze the addition of activated sugars (donor NDP-sugars), in a step-wise fashion, to a substrate (e.g., protein, glycopeptide, lipid, glycolipid or to the non-reducing end of a growing oligosaccharide). A very large number of glycosyltransferases are known in the art.

Using the methods of the invention, it is possible to prepare immobilized glycosyltransferases that are selected to have a desired specificity. The glycosyltransferases preferably also are capable of glycosylating a high percentage of a selected acceptor group of the substrate. The SBD can be conjugated to the enzyme or it can be a component of a fusion protein that includes a SBD peptide sequence. Other glycosyltransferase fusion proteins include glycosyltransferases that exhibit the activity of two different glycosyltransferases (e.g., sialyltransferase and fucosyltransferase). Other fusion proteins will include two different variations of the same transferase activity (e.g., FucT-VI and FucT-VII). Still other fusion proteins will include a domain that enhances the utility of the transferase activity (e.g, enhanced solubility, stability, turnover, etc.).

The SBD-containing glycosyltransferase can be used to prepare a selected glycosyl moiety. A number of methods of using glycosyltransferases to synthesize desired oligosaccharide structures are known and are generally applicable to the instant invention. Exemplary methods are described, for instance, WO 96/32491, Ito et al., Pure Appl. Chem. 65: 753 (1993), and U.S. Pat. Nos. 5,352,670, 5,374,541, and 5,545,553.

The method of the invention may utilize any glycosyltransferase, provided that it adds a desired glycosyl residue at a selected site. Examples of such enzymes include fucosyltransferase, sialyltransferase, mannosyltransferase, xylosyltransferase, glucosyltransferase, glucurononyltransferase and the like.

Glycosyltransferases that can be employed in the methods of the invention include, but are not limited to, galactosyltransferases, fucosyltransferases, glucosyltransferases, N-acetylgalactosaminyltransferases, N-acetylglucosaminyltransferases, glucuronyltransferases, sialyltransferases, mannosyltransferases, glucuronic acid transferases, and galacturonic acid transferases. Suitable glycosyltransferases include those obtained from eukaryotes, as well as from prokaryotes.

For enzymatic saccharide syntheses that involve glycosyltransferase reactions, glycosyltransferase can be cloned, or isolated from any source. Many cloned glycosyltransferases are known, as are their polynucleotide sequences. See, e.g., “The WWW Guide To Cloned Glycosyltransferases,” (http://www.vei.co.uk/TGN/gt guide.htm). Glycosyltransferase amino acid sequences and nucleotide sequences encoding glycosyltransferases from which the amino acid sequences can be deduced are also found in various publicly available databases, including GenBank, Swiss-Prot, EMBL, and others.

DNA encoding the glycosyltransferases may be obtained by chemical synthesis, by screening reverse transcripts of mRNA from appropriate cells or cell line cultures, by screening genomic libraries from appropriate cells, or by combinations of these procedures. Screening of mRNA or genomic DNA may be carried out with oligonucleotide probes generated from the glycosyltransferases gene sequence. Probes may be labeled with a detectable group such as a fluorescent group, a radioactive atom or a chemiluminescent group in accordance with known procedures and used in conventional hybridization assays. In the alternative, glycosyltransferases gene sequences may be obtained by use of the polymerase chain reaction (PCR) procedure, with the PCR oligonucleotide primers being produced from the glycosyltransferases gene sequence. See, U.S. Pat. No. 4,683,195 to Mullis et al. and U.S. Pat. No. 4,683,202 to Mullis.

The glycosyltransferase may be synthesized in host cells transformed with vectors containing DNA encoding the glycosyltransferase. A vector is a replicable DNA construct. Vectors are used either to amplify DNA encoding the glycosyltransferases enzyme and/or to express DNA, which encodes the glycosyltransferases enzyme. An expression vector is a replicable DNA construct in which a DNA sequence encoding the glycosyltransferases the glycosyltransferase in a suitable host. The need for such control sequences will vary depending upon the host selected and the transformation method chosen. Generally, control sequences include a transcriptional promoter, an optional operator sequence to control transcription, a sequence encoding suitable mRNA ribosomal binding sites, and sequences that control the termination of transcription and translation. Amplification vectors do not require expression control domains. All that is needed is the ability to replicate in a host, usually conferred by an origin of replication, and a selection gene to facilitate recognition of transformants.

Examples of suitable glycosyltransferases for use in the preparation of the compositions of the invention are described herein. One can readily identify other suitable glycosyltransferases by reacting various amounts of each enzyme (e.g., 1-100 mU/mg protein) with a substrate (e.g., at 1-10 mg/ml) to which is linked an oligosaccharide that has a potential acceptor site for the glycosyltransferase of interest. The abilities of the glycosyltransferases to add a sugar residue at the desired site are compared. Glycosyltransferases showing the ability to glycosylate the potential acceptor sites of substrate-linked oligosaccharides more efficiently than other glycosyltransferases having the same specificity are suitable for use in the methods of the invention.

The amount of a particular enzyme needed to accomplish a desired transformation is readily determined by those of skill in the art. In other embodiments, however, it is desirable to use a greater amount of enzyme. A temperature of about 30 to about 37° C., for example, is suitable.

The efficacy of the methods of the invention can be enhanced through use of recombinantly produced glycosyltransferases. Recombinant production enables production of glycosyltransferases in the large amounts that are required for large-scale substrate modification. Deletion of the membrane-anchoring domain of glycosyltransferases, which renders the glycosyltransferases soluble and thus facilitates production and purification of large amounts of glycosyltransferases, can be accomplished by recombinant expression of a modified gene encoding the glycosyltransferases. For a description of methods suitable for recombinant production of glycosyltransferases see, U.S. Pat. No. 5,032,519.

Also provided by the invention are glycosylation methods in which the target substrate is immobilized on a solid support. The term “solid support” also encompasses substrate can be released after the glycosylation reaction is completed. Suitable matrices are known to those of skill in the art. Ion exchange, for example, can be employed to temporarily immobilize a substrate on an appropriate resin while the glycosylation reaction proceeds. A ligand that specifically binds to the substrate of interest can also be used for affinity-based immobilization. Antibodies that bind to a substrate of interest are suitable. Dyes and other molecules that specifically bind to a substrate of interest that is to be glycosylated are also suitable.

Other exemplary enzymes of use in the present invention include fucosyltransferases. Many saccharides require the presence of particular fucosylated structures in order to exhibit biological activity. Intercellular recognition mechanisms often require a fucosylated oligosaccharide. For example, a number of proteins that function as cell adhesion molecules, including P-selectin, E-selectin, bind specific cell surface fucosylated carbohydrate structures, for example, the sialyl Lewis x and the sialyl Lewis a structures. In addition, the specific carbohydrate structures that form the ABO blood group system are fucosylated. The carbohydrate structures in each of the three groups share a Fucα1,2Galβ1-dissacharide unit. In blood group O structures, this disaccharide is the terminal structure. The group A structure is formed by an α1,3 GalNAc transferase that adds a terminal GalNAc residue to the dissacharide. The group B structure is formed by an α1,3 galactosyltransferase that adds terminal galactose residue. The Lewis blood group structures are also fucosylated. For example the Lewis x and Lewis a structures are Galβ1,4(Fucα1,3)GlcNac and Galβ1,4(Fucα1,4)GlcNac, respectively. Both these structures can be further sialylated (NeuAcα2,3-) to form the corresponding sialylated structures. Other Lewis blood group structures of interest are the Lewis y and b structures which are Fucα1,2Galβ1,4(Fucα1,3)GlcNAcβ-OR and Fucα1,2Galβ1,3(Fucα1,4)GlcNAc-OR, respectively. For a description of the structures of the ABO and Lewis blood group stuctures and the enzymes involved in their synthesis see, Essentials of Glycobiology, Varki et al. eds., Chapter 16 (Cold Spring Harbor Press, Cold Spring Harbor, N.Y., 1999).

Fucosyltransferases have been used in synthetic pathways to transfer a fucose unit from guanosine-5′-diphosphofucose to a specific hydroxyl of a saccharide acceptor. For example, Ichikawa prepared sialyl Lewis-X by a method that involves the fucosylation of sialylated lactosamine with a cloned fucosyltransferase (Ichikawa et al., J. Am. Chem. Soc. [symbol] fucosylation activity in cells, thereby producing fucosylated glycoproteins, cell surfaces, etc. (U.S. Pat. No. 5,955,347).

In one embodiment, the methods of the invention are practiced by contacting a substrate, having an acceptor moiety for a fucosyltransferase, with a reaction mixture that includes a fucose donor moiety, a fucosyltransferase, and other reagents required for fucosyltransferase activity. The substrate is incubated in the reaction mixture for a sufficient time and under appropriate conditions to transfer fucose from the fucose donor moiety to the fucosyltransferase acceptor moiety. In preferred embodiments, the fucosyltransferase catalyzes the fucosylation of at least 60% of the fucosyltransferase respective acceptor moieties in the composition.

A number of fucosyltransferases are known to those of skill in the art. Briefly, fucosyltransferases include any of those enzymes, which transfer L-fucose from GDP-fucose to a hydroxy position of an acceptor sugar. In some embodiments, for example, the acceptor sugar is a GlcNAc in a Galβ(1→3,4)GlcNAc group in an oligosaccharide glycoside. Suitable fucosyltransferases for this reaction include the known Galβ(1→3,4)GlcNAc α(1→3,4)fucosyltransferase (FucT-III E.C. No. 2.4.1.65) which is obtained from human milk (see, e.g., Palcic et al., Carbohydrate Res. 190:1-11 (1989); Prieels, et al., J. Biol. Chem. 256:10456-10463 (1981); and Nunez, et al., Can. J. Chem. 59:2086-2095 (1981)) and the βGal(1→4)βGlcNAc α(1→3,4)fucosyltransferases (FucT-IV, FucT-V, FucT-VI, and FucT-VII, E.C. No. 2.4.1.65) which are found in human serum. A recombinant form of βGal(1→3,4)βGlcNAc α(1→3,4)fucosyltransferase is also available (see, Dumas, et al., Bioorg. Med. Letters 1: 425428 (1991) and Kukowska-Latallo, et al., Genes and Development 4: 1288-1303 (1990)). Other exemplary fucosyltransferases include α1,2 fucosyltransferase (E.C. No. 2.4.1.69). Enzymatic fucosylation may be carried out by the methods described in Mollicone et al., Eur. J. Biochem. 191:169-176 (1990) or U.S. Pat. No. 5,374,655; an α1,3-fucosyltransferase from Schistosoma mansoni (Trottein et al. (2000) Mol. Biochem. Parasitol. 107: 279-287); and an α1,3 fucosyltransferase IX (nucleotide sequences of human and mouse FucT-IX are described in Kaneko et al. (1999) FEBS Lett. 452: 237-242, and the chromosomal location of the human gene is described in Kaneko et al. (1999) Cytogenet. Cell Genet. 86: 329-330. Recently reported α1,3-fucosyltransferases that use an N-linked GlcNAc as an acceptor from the snail Lymnaea stagnalis and from mung (1999) J. Biol. Chem. 274: 21830-21839, respectively. In addition, bacterial fucosyltransferases such as the α(1,3/4) fucosyltransferase of Helicobacter pylori as described in Rasko et al. (2000) J. Biol. Chem. 275:4988-94, as well as the α1,2-fucosyltransferase of H. Pylori (Wang et al. (1999) Microbiology. 145: 3245-53. See, also Staudacher, E. (1996) Trends in Glycoscience and Glycotechnology, 8: 391-408 for description of fucosyltransferases useful in the invention.

Suitable acceptor moieties for fucosyltransferase-catalyzed attachment of a fucose residue include, but are not limited to, GlcNAc-OR, Galβ1,3GlcNAc-OR, NeuAcα2,3Galβ1,3GlcNAc-OR, Galβ1,4GlcNAc-OR and NeuAcα2,3Galβ1,4GlcNAc-OR, where R is an amino acid, a saccharide, an oligosaccharide or an a glycon group having at least one carbon atom. R is linked to or is part of a substrate. The appropriate fucosyltransferase for a particular reaction is chosen based on the type of fucose linkage that is desired (e.g., α2, α3, or α4), the particular acceptor of interest, and the ability of the fucosyltransferase to achieve the desired high yield of fucosylation. Suitable fucosyltransferases and their properties are described above.

If a sufficient proportion of the substrate-linked oligosaccharides in a composition does not include a fucosyltransferase acceptor moiety, one can synthesize a suitable acceptor. For example, one preferred method for synthesizing an acceptor for a fucosyltransferase involves use of a GlcNAc transferase to attach a GlcNAc residue to a GlcNAc transferase acceptor moiety, which is present on the substrate-linked oligosaccharides. In preferred embodiments a transferase is chosen, having the ability to glycosylate a large fraction of the potential acceptor moieties of interest. The resulting GlcNAcβ-OR can then be used as an acceptor for a fucosyltransferase.

The resulting GlcNAcβ-OR moiety can be galactosylated prior to the fucosyltransferase reaction, yielding, for example, a Galβ1,3GlcNAc-OR or Gal β1,4GlcNAc-OR residue. In some embodiments, the galactylation and fucosylation steps can be carried out simultaneously. By choosing a fucosyltransferase that requires the galactosylated acceptor, only the desired product is formed. Thus, this method involves: [symbol] galactosyltransferase in the presence of a UDP-galactose under conditions sufficient to form the compounds Galβ1,4GlcNAcβ-OR or Galβ1,3GlcNAc-OR; and
(b) fucosylating the compound formed in (a) using a fucosyltransferase in the presence of GDP-fucose under conditions sufficient to form a compound selected from:
   Fucα1,2Galβ1,4GlcNAc1β-O1R;
   Fucα1,2Galβ1,3GlcNAc-OR;
   Fucα1,2Galβ1,4GalNAc1β-O1R;
   Fucα1,2Galβ1,3GalNAc-OR;
   Galβ1,4(Fucα1,3)GlcNAcβ-OR; or
   Galβ1,3(Fucα1,4)GlcNAc-OR.

One can add additional fucose residues to the above structures by including an additional fucosyltransferase, which has the desired activity. For example, the methods can form oligosaccharide determinants such as Fucα1,2Galβ1,4(Fucα1,3)GlcNAcβ-OR and Fucα1,2Galβ1,3(Fucα1,4)GlcNAc-OR. Thus, in another preferred embodiment, the method includes the use of at least two fucosyltransferases. The multiple fucosyltransferases are used either simultaneously or sequentially. When the fucosyltransferases are used sequentially, it is generally preferred that the glycoprotein is not purified between the multiple fucosylation steps. When the multiple fucosyltransferases are used simultaneously, the enzymatic activity can be derived from two separate enzymes or, alternatively, from a single enzyme having more than one fucosyltransferase activity.

Sialyltransferases

The methods of the invention can also be practiced using a SBD-tagged sialyltransferase. Examples of recombinant sialyltransferases, including those having deleted anchor domains, as well as methods of producing recombinant sialyltransferases, are found in, for example, U.S. Pat. No. 5,541,083. At least 15 different mammalian sialyltransferases have been documented, and the cDNAs of thirteen of these have been cloned to date (for the systematic nomenclature that is used herein, see, Tsuji et al. (1996) Glycobiology 6: v-xiv). [symbol] be used in the methods of the invention.

The sialylation can be accomplished using either a trans-sialidase or a sialyltransferase, except where a particular determinant requires an α2,6-linked sialic acid, in which case a sialyltransferase is used. The present methods involve sialylating an acceptor for a sialyltransferase or a trans-sialidase by contacting the acceptor with the appropriate enzyme in the presence of an appropriate donor moiety. For sialyltransferases, CMP-sialic acid is a preferred donor moiety. Trans-sialidases, however, preferably use a donor moiety that includes a leaving group to which the trans-sialidase cannot add sialic acid.

Acceptor moieties of interest include, for example, Galβ-OR. In some embodiments, the acceptor moieties are contacted with a sialyltransferase in the presence of CMP-sialic acid under conditions in which sialic acid is transferred to the non-reducing end of the acceptor moiety to form the compound NeuAcα2,3Galβ-OR or NeuAcα2,6Galβ-OR. In this formula, R is an amino acid, a saccharide, an oligosaccharide or an a glycon group having at least one carbon atom. In an exemplary embodiment, Galβ-OR is Galβ1,4GlcNAc-R, wherein R is linked to or is part of a substrate.

In an exemplary embodiment, the method provides a compound that is both sialylated and fucosylated. The sialyltransferase and fucosyltransferase reactions are generally conducted sequentially, since most sialyltransferases are not active on a fucosylated acceptor. FucT-VII, however, acts only on a sialylated acceptor. Therefore, FucT-VII can be used in a simultaneous reaction with a sialyltransferase.

If the trans-sialidase is used to accomplish the sialylation, the fucosylation and sialylation reactions can be conducted either simultaneously or sequentially, in either order. The substrate to be modified is incubated with a reaction mixture that contains a suitable amount of a trans-sialidase, a suitable sialic acid donor substrate, a fucosyltransferase (capable of making an α1,3 or α1,4 linkage), and a suitable fucosyl donor substrate (e.g., GDP-fucose).

Examples of sialyltransferases that are suitable for use in the present invention include ST3Gal III (e.g., a rat or human ST3Gal III), ST3Gal IV, ST3Gal I, ST6Gal I, ST3Gal V, ST6Gal II, ST6GalNAc I, ST6GalNAc II, and ST6GalNAc III (the sialyltransferase nomenclature used herein is as described in Tsuji et al., Glycobiology 6: v- [symbol] 2.4.99.6) transfers sialic acid to the non-reducing terminal Gal of a Galβ1→3Glc disaccharide or glycoside. See, Van den Eijnden et al., J. Biol. Chem. 256: 3159 (1981), Weinstein et al., J. Biol. Chem. 257: 13845 (1982) and Wen et al., J. Biol. Chem. 267: 21011 (1992). Another exemplary α2,3-sialyltransferase (EC 2.4.99.4) transfers sialic acid to the non-reducing terminal Gal of the disaccharide or glycoside. see, Rearick et al., J. Biol. Chem. 254: 4444 (1979) and Gillespie et al., J. Biol. Chem. 267: 21004 (1992). Further exemplary enzymes include Gal-β-1,4-GlcNAc α-2,6 sialyltransferase (See, Kurosawa et al. Eur. J. Biochem. 219: 375-381 (1994)). An α2,8-sialyltransferase can also be used to attach a second or multiple sialic acid residues to substrates useful in methods of the invention. A still further example is the alpha2,3-sialyltransferases from Streptococcus agalactiae (ST known as cpsK gene), Haemophilus ducreyi (known as 1st gene), Haemophilus influenza (known as HI0871 gene). See, Chaffin et al., Mol. Microbiol., 45: 109-122 (2002).

An example of a sialyltransferase that is useful in the claimed methods is CST-I from Campylobacter (see, for example, U.S. Pat. Nos. 6,503744, 6,096,529, and 6,210933 and WO99/49051, and published U.S. Pat. Application 2002/2,042,369). This enzyme catalyzes the transfer of sialic acid to the Gal of a Galβ1,4Glc or Galβ1,3GalNAc

Other exemplary sialyltransferases of use in the present invention include those isolated from Campylobacter jejuni, including the α(2,3) sialyltransferase. See, e.g, WO99/49051. In another embodiment, the invention provides bifunctional sialyltransferase polypeptides that have both an α2,3 sialyltransferase activity and an α2,8 sialyltransferase activity. The bifunctional sialyltransferases, when placed in a reaction mixture with a suitable saccharide acceptor (e.g., a saccharide having a terminal galactose), and a sialic acid donor (e.g., CMP-sialic acid) can catalyze the transfer of a first sialic acid from the donor to the acceptor in an α2,3 linkage. The sialyltransferase then catalyzes the transfer of a second sialic acid from a sialic acid donor to the first sialic acid residue in an α2,8 linkage. This type of Siaα2,8-Siaα2,3-Gal structure is often found in glycosphingolipids. See, for example, EP Pat. App. No. 1147200.

A recently reported viral α2,3-sialyltransferase is also suitable use in the sialylation methods of the invention (Sujino et al. (2000) Glycobiology 10: 313-320). This enzyme, v-ST3Gal I, was obtained from Myxoma virus-infected cells and is apparently related to the mammalian ST3Gal IV as indicated by comparison of the respective amino acid sequences. [symbol] (Galβ1,4GlcNAc-β1-R) and III (Galβ1,3GalNAcβ1-R) acceptors. The enzyme can also transfer sialic acid to fucosylated acceptor moieties (e.g., Lewis^{x} and Lewis^{a}).

Galactosyltransferases

In another group of embodiments, the SBD-tagged enzyme is a glycosyltransferase. Exemplary galactosyltransferases include o:(1,3) galactosyltransferases (E.C. No. 2.4.1.151, see, e.g., Dabkowski et al., Transplant Proc. 25:2921 (1993) and Yamamoto et al. Nature 345: 229-233 (1990), bovine (GenBankj04989, Joziasse et al., J. Biol. Chem. 264: 14290-14297 (1989)), murine (GenBank m26925; Larsen et al., Proc. Nat'l. Acad. Sci. USA 86: 8227-8231 (1989)), porcine (GenBank L36152; Strahan et al., Immunogenetics 41: 101-105 (1995)). Another suitable α1,3 galactosyltransferase is that which is involved in synthesis of the blood group B antigen (EC 2.4.1.37, Yamamoto et al., J. Biol. Chem. 265: 1146-1151 (1990) (human)). The present invention can also be practiced using α1,4-galactosyltransferases.

Also suitable for use in the methods of the invention are β(1,4) galactosyltransferases, which include, for example, EC 2.4.1.90 (LacNAc synthetase) and EC 2.4.1.22 (lactose synthetase) (bovine (D'Agostaro et al., Eur. J. Biochem. 183: 211-217 (1989)), human (Masri et al., Biochem. Biophys. Res. Commun. 157: 657-663 (1988)), murine (Nakazawa et al., J. Biochem. 104: 165-168 (1988)), as well as E.C. 2.4.1.38 and the ceramide galactosyltransferase (EC 2.4.1.45, Stahl et al., J. Neurosci. Res. 38: 234-242 (1994)). Other suitable galactosyltransferases include, for example, α1,2 galactosyltransferases (from e.g., Schizosaccharomyces pombe, Chapell et al., Mol. Biol. Cell 5: 519-528 (1994)). Other 1,4-galactosyltransferases are those used to produce globosides (see, for example, Schaeper, et al. Carbohydrate Research 1992, vol. 236, pp. 227-244. Both mammalian and bacterial enzymes are of use.

Other exemplary galactosyltransferases of use in the invention include β1,3-galactosyltransferases. When placed in a suitable reaction medium, the β1,3-galactosyltransferases, catalyze the transfer of a galactose residue from a donor (e.g., UDP-Gal) to a suitable saccharide acceptor (e.g., saccharides having a terminal GalNAc residue). An example of a β1,3-galactosyltransferase of the invention is that produced by [symbol] of the invention is that of C. jejuni strain OH4384 as

Exemplary linkages in compounds formed by the method of the invention using galactosyltransferases include: (1) Galβ1→4Glc; (2) Galβ1→4GlcNAc; (3) Galβ1→3GlcNAc; (4) Gal1→6GlcNAc; (5) Galβ1→3GalNAc; (6) Galβ1→6GalNAc; (7) Galα1→3GalNAc; (8) Galα1→3Gal; (9) Galα1→4Gal; (10) Galβ1→3Gal; (11) Galβ1→4Gal; (12) Galβ1→6Gal; (13) Galβ1→4xylose; (14) Galβ1→1′-sphingosine; (15) Galβ1→1′-ceramide; (16) Galβ1→3 diglyceride; (17) Galβ1→O-hydroxylysine; and (18) Gal-S-cysteine. See, for example, U.S. Pat. Nos. 6,268,193; and 5,691,180.

Trans-sialidase

The method of the invention can also be practiced using a SBD-tagged trans-sialidase. As used herein, the term “trans-sialidase” refers to an enzyme that catalyzes the addition of a sialic acid to galactose through an α-2,3 glycosidic linkage. Trans-sialidases are found in many Trypanosomy species and some other parasites. Trans-sialidases of these parasite organisms retain the hydrolytic activity of usual sialidase, but with much less efficiency, and catalyze a reversible transfer of terminal sialic acids from host sialoglycoconjugates to parasite surface glycoproteins in the absence of CMP-sialic acid. Trypanosome cruzi, which causes Chagas disease, has a surface trans-sialidase the catalyzes preferentially the transference of α-2,3-linked sialic acid to acceptors containing terminal β-galactosyl residues, instead of the typical hydrolysis reaction of most sialidases (Ribeirao et al., Glycobiol. 7: 1237-1246 (1997); Takahashi et al., Anal. Biochem. 230: 333-342 (1995); Scudder et al., J. Biol. Chem. 268: 9886-9891 (1993); and Vandekerckhove et al., Glycobiol. 2: 541-548 (1992)). T. cruzi trans-sialidase (TcTs) has activity towards a wide range of saccharide, glycolipid, and glycoprotein acceptors which terminate with a β-linked galactose residue, and synthesizes exclusively an α2-3 sialosidic linkage (Scudder et al., supra). At a low rate, it also transfers sialic acid from synthetic α-sialosides, such as p-nitrophenyl-α-N-acetylneuraminic acid, but NeuAc2-3Galβ1-4(Fucα1-3)Glc is not a donor-substrate. Modified 2-[4-methylumbelliferone]-α-ketoside of N-acetyl-D-neuraminic acid (4MU-NANA) and several derivatives thereof can also serve as donors for TcTs (Lee & Lee, Anal. Biochem. 216: 358-364 (1994)). Enzymatic synthesis of 3′-sialyl-lacto-N-biose I has been catalyzed by TcTs from lacto-N-biose I as acceptor and 2′-(4-methylumbellyferyl)-α-D-N-acelyneuraminic as donor of the N-acetylneuraminil moiety (Vetere et al., Eur. J. Biochem. α2,3-sialylated conjugates can be found in European Patent Application No. 0 557 580 A2 and U.S. Pat. No. 5,409,817, each of which is incorporated herein by reference. The intramolecular trans-sialidase from the leech Macrobdella decora exhibits strict specificity toward the cleavage of terminal Neu5Ac (N-acetylneuraminic acid) α2→3Gal linkage in sialoglycoconjugates and catalyzes an intramolecular trans-sialosyl reaction (Luo et al., J. Mol. Biol. 285: 323-332 (1999). Trans-sialidases primarily add sialic acid onto galactose acceptors, although, they will transfer sialic acid onto some other sugars. Transfer of sialic acid onto GalNAc, however, requires a sialyltransferase. Further information on the use of trans-sialidases can be found in PCT Application No. WO 93/18787; and Vetere et al., Eur. J. Biochem. 247: 1083-1090 (1997).

GalNAc Transferases

The invention also may also utilize a SBD-tagged β1,4-GalNAc transferase polypeptides. The β1,4-GalNAc transferases, when placed in a reaction mixture, catalyze the transfer of a GalNAc residue from a donor (e.g., UDP-GalNAc) to a suitable acceptor saccharide (typically a saccharide that has a terminal galactose residue). The resulting structure, GalNAcβ1,4-Gal-, is often found in glycosphingolipids and other sphingoids, among many other saccharide compounds.

An example of a β1,4-GalNAc transferase useful in the present invention is that produced by Campylobacter species, such as C. jejuni. A presently preferred β1,4-GalNAc transferase polypeptide is that of C. jejuni strain OH4384.

Exemplary GalNAc transferases of use in the present invention form the following linkages: (1) (GalNAcα1-3)[(Fucα1-2)]Galβ-; (2) GalNAcα1→Ser/Thr; (3) GalNAcβ1→4Gal; (4) GalNAcβ1→3Gal; (5) GalNAcα1→3GalNAc; (6) (GalNAcβ1→4GlcUAβ1→3)ₙ; (7) (GalNAcβ1→4ldUAα1→3-)ₙ; (8) -Manβ-4GalNAcαGlcNAcαAsn. See, for example, U.S. Pat. Nos. 6,268,193; and 5,691,180.

GlcNAc Transferases

In yet another exemplary embodiment, the invention makes use of a SBD-tagged GlcNAc transferase. Exemplary N-Acetylglucosaminyltransferases useful in practicing the present invention are able to form the following linkages: (1) GlcNAcβ1→4GlcNAc; (2) GlcNAcβ1→3Man; (7) GlcNAcα1→3Man; (8) GlcNAcβ1→.3Gal; (9) GlcNAcβ1→4Gal; (10) GlcNAcβ1→6Gal; (11) GlcNAcα1→4Gal; (12) GlcNAcα1→4GlcNAc; (13) GlcNAcβ1→6GalNAc; (14) GlcNAcβ1→3GalNAc; (15) GlcNAcβ→4GlcUA; (16) GlcNAcα1→4GlcUA; (17) GlcNAcα1→4IdUA. See, for example, U.S. Pat. Nos. 6,268,193; and 5,691,180.

Other Glycosyltransferases

Other SBD-tagged glycosyltransferases can be substituted into similar transferase cycles as have been described in detail for the fucosyltransferases and sialyltransferases. In particular, the glycosyltransferase can also be, for instance, glucosyltransferases, e.g., Alg8 (Stagljov et al., Proc. Natl. Acad. Sci. USA 91:5977 (1994)) or Alg5 (Heesen et al. Eur. J. Biochem. 224:71 (1994)), N-acetylgalactosaminyltransferases such as, for example, c(1,3) N-acetylgalactosaminyltransferase, β(1,4) N-acetylgalactosaminyltransferases (Nagata et al. J. Biol. Chem. 267:12082-12089 (1992) and Smith et al. J. Biol Chem. 269:15162 (1994)) and polypeptide N-acetylgalactosaminyltransferase (Homa et al. J. Biol Chem. 268:12609 (1993)). Suitable N-acetylglucosaminyltransferases include GnTI (2.4.1.101, Hull et al., BBRC 176:608 (1991)), GnTII, and GnTIII (Ihara et al. J. Biochem. 113:692 (1993)), GnTV (Shoreiban et al. J. Biol. Chem. 268: 15381 (1993)), O-linked N-acetylglucosaminyltransferase (Bierhuizen et al. Proc. Natl. Acad. Sci. USA 89:9326 (1992)), N-acetylglucosamine-1-phosphate transferase (Rajput et al. Biochem J. 285:985 (1992), and hyaluronan synthase. Suitable mannosyltransferases include α(1,2) mannosyltransferase, α(1,3) mannosyltransferase, β(1,4) mannosyltransferase, Dol-P-Man synthase, OCh1, and Pmt1.

Cloning Of Glycosyltransferases And Recombinant Glycosyltransferase Fusion Proteins

In an exemplary embodiment, the invention utilizes a fusion protein that includes a SBD encoded in its peptide sequence. The present invention is exemplified by polypeptide species that are of use to perform synthetic transformation, including enzymes such as glycosyltransferases. The focus on fusion proteins of glycosyltransferases is for clarity of illustration and those of skill in the art will appreciate that the practice of the present invention is not limited to the use of enzymes in general or glycosyltransferases specifically. [symbol] nucleic acids, are known to those of skill in the art. Suitable nucleic acids (e.g. cDNA, genomic, or subsequences (probes)) can be cloned, or amplified by in vitro methods such as the polymerase chain reaction (PCR), the ligase chain reaction (LCR), the transcription-based amplification system (TAS), or the self-sustained sequence replication system (SSR). A wide variety of cloning and in vitro amplification methodologies are well-known to persons of skill. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, N.Y., (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864.

DNA that encodes a glycosyltransferase, or a subsequence thereof, can be prepared by any suitable method described above, including, for example, cloning and restriction of appropriate sequences with restriction enzymes. In one preferred embodiment, nucleic acids encoding glycosyltransferases are isolated by routine cloning methods. A nucleotide sequence of a glycosyltransferase as provided in, for example, GenBank or other sequence database (see above) can be used to provide probes that specifically hybridize to a glycosyltransferase gene in a genomic DNA sample, or to an mRNA, encoding a glucosyltransferase, in a total RNA sample (e.g., in a Southern or Northern blot). Once the target nucleic acid encoding a glycosyltransferase is identified, it can be isolated according to standard methods known to those of skill in the art (see, e.g., Sambrook et al. (1989) Molecular Cloning: A Laboratory Manual, 2nd Ed., Vols. 1-3, Cold Spring Harbor Laboratory; Berger and Kimmel (1987) Methods in Enzymology, Vol. 152: Guide to Molecular Cloning Techniques, San Diego: Academic Press, Inc.; or Ausubel et al. (1987) Current Protocols in Molecular Biology, Greene Publishing and Wiley-Interscience, New York). Further, the isolated nucleic acids can be cleaved with restriction enzymes to create nucleic acids encoding the full-length glycosyltransferse, or subsequences thereof, e.g., containing subsequences encoding at least a subsequence of a stem region or catalytic domain of a glycosyltransferase. These restriction enzyme fragments, encoding a glycosyltransferase [symbol] encoding a recombinant glycosyltransferase fusion protein.

A nucleic acid encoding a glycosyltransferase, or a subsequence thereof, can be characterized by assaying for the expressed product. Assays based on the detection of the physical, chemical, or immunological properties of the expressed protein can be used. For example, one can identify a cloned glycosyltransferase, including a glycosyltransferase fusion protein, by the ability of a protein encoded by the nucleic acid to catalyze the transfer of a saccharide from a donor substrate to an acceptor substrate. In a preferred method, capillary electrophoresis is employed to detect the reaction products. This highly sensitive assay involves using either saccharide or disaccharide aminophenyl derivatives which are labeled with fluorescein as described in Wakarchuk et al. (1996) J. Biol. Chem. 271 (45): 28271-276. For example, to assay for a Neisseria lgtC enzyme, either FCHASE-AP-Lac or FCHASE-AP-Gal can be used, whereas for the Neisseria lgtB enzyme an appropriate reagent is FCHASE-AP-GlcNAc (Id.).

Also, a nucleic acid encoding a glycosyltransferase, or a subsequence thereof, can be chemically synthesized. Suitable methods include the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill recognizes that while chemical synthesis of DNA is often limited to sequences of about 100 bases, longer sequences may be obtained by the ligation of shorter sequences.

Nucleic acids encoding glycosyltransferases, or subsequences thereof, can be cloned using DNA amplification methods such as polymerase chain reaction (PCR). Thus, for example, the nucleic acid sequence or subsequence is PCR amplified, using a sense primer containing one restriction enzyme site (e.g., NdeI) and an antisense primer containing another restriction enzyme site (e.g., HindIII). This will produce a nucleic acid encoding the desired glycosyltransferase or subsequence and having terminal restriction enzyme sites. This nucleic acid can then be easily ligated into a vector containing a nucleic acid encoding the second molecule and having the appropriate corresponding restriction enzyme sites. Suitable [symbol] provided in GenBank or other sources. Appropriate restriction enzyme sites can also be added to the nucleic acid encoding the glycosyltransferase protein or protein subsequence by site-directed mutagenesis. The plasmid containing the glycosyltransferase-encoding nucleotide sequence or subsequence is cleaved with the appropriate restriction endonuclease and then ligated into an appropriate vector for amplification and/or expression according to standard methods. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

Other physical properties of a cloned glycosyltransferase protein, including glycosyltransferase fusion protein, expressed from a particular nucleic acid, can be compared to properties of known glycosyltransferases to provide another method of identifying suitable sequences or domains of the glycosyltransferase that are determinants of acceptor substrate specificity and/or catalytic activity. Alternatively, a putative glycosyltransferase gene or recombinant glycosyltransferase gene can be mutated, and its role as glycosyltransferase, or the role of particular sequences or domains established by detecting a variation in the structure of a carbohydrate normally produced by the unmutated, naturally-occurring, or control glycosyltransferase.

Functional domains of cloned glycosyltransferases can be identified by using standard methods for mutating or modifying the glycosyltransferases and testing the modified or mutated proteins for activities such as acceptor substrate activity and/or catalytic activity, as described herein. The functional domains of the various glycosyltransferases can be used to construct nucleic acids encoding recombinant glycosyltransferase fusion proteins comprising the functional domains of one or more glycosyltransferases. These fusion proteins can then be tested for the desired acceptor substrate or catalytic activity. [symbol] proteins, the known nucleic acid or amino acid sequences of cloned glycosyltransferases are aligned and compared to determine the amount of sequence identity between various glycosyltransferases. This information can be used to identify and select protein domains that confer or modulate glycosyltransferase activities, e.g., acceptor substrate activity and/or catalytic activity based on the amount of sequence identity between the glycosyltransferases of interest. For example, domains having sequence identity between the glycosyltransferases of interest, and that are associated with a known activity, can be used to construct recombinant glycosyltransferase fusion proteins containing that domain, and having the activity associated with that domain (e.g., acceptor substrate specificity and/or catalytic activity).

Fusion proteins of the invention can be expressed in a variety of host cells, including E. coli, other bacterial hosts, yeast, and various higher eukaryotic cells such as the COS, CHO and HeLa cells lines and myeloma cell lines. The host cells can be mammalian cells, plant cells, or microorganisms, such as, for example, yeast cells, bacterial cells, or filamentous fungal cells. Examples of suitable host cells include, for example, Azotobacter sp. (e.g., A. vinelaitdii), Pseudomonas sp., Rhizobium sp., Erwinia sp., Escherichia sp. (e.g., E. coli), Bacillus, Pseudomonas, Proteus, Salmonella, Serratia, Shigella, Rhizobia, Vitreoscilla, Paracoccus and Klebsiella sp., among many others. The cells can be of any of several genera, including Saccharomyces (e.g., S. cerevisiae), Candida (e.g., C. utilis, C. parapsilosis, C. krusei, C. versatilis, C. lipolytica, C. zeylanoides, C. guillierinondii, C. albicans, and C. humicola), Pichia (e.g., P. farinosa and P. ohmeri), Torulopsis (e.g., T candida, T. sphaerica, T. xylinus, T. famata, and T. versatilis), Debaryomyces (e.g., D. subglobosus, D. cantarellii, D. globosus, D. hansenii, and D. japonicus), Zygosaccharomyces (e.g., Z. rouxii and Z. bailii), Kluyveromyces (e.g., K marxianus), Hansenula (e.g., H. anomala and H. jadinii), and Brettanomyces (e.g., B. lambicus and B. anomalus). Examples of useful bacteria include, but are not limited to, Escherichia, Enterobacter, Azotobacter,; Erwinia, Klebsielia.

Examples of a fungal host cell are filamentous fungal cell. “Filamentous fungi” include all filamentous forms of the subdivision Eumycota and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. In [symbol] unicellular thallus and carbon catabolism may be fermentative.

More particularly, the filamentous fungal host cell is a cell of a species of, but not limited to, Acremonium, Aspergillus, Fusarium, Humicola, Mucor, Myceliophthora, Neurospora, Penicillium, Phanerochaeta, Thielavia, Tolypocladium, or Trichoderma. In a preferred embodiment, the filamentous fungal host cell is, but not limited to, an Aspergillus niger, Aspergillus awamori, Aspergillus foetidus, Aspergillus japonicus, Aspergillus nidulans, or Aspergillus oryzae cell. Other examples of suitable filamentous fungal host cells are Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinunm, Fusarium sarcochroum, Fusarium sporotrichioides, Fusarium sulphureum, Fusarium torulosum, Fusarium trichothecioides, or Fusarium venenatum cells. Also suitable is the filamentous fungal cell is a Fusarium venenatum (Nirenberg sp. nov.) cell. Further examples of suitable filamentous fungal host cells are Humicola insolens, Humicola lanuginosa, Mucor miehei, Myceliophthora thermophila, Neurospora crassa, Penicillium purpurogenum, Phanerochaeta chrysosporium, Thielavia terrestris, Trichoderma harzianum, Trichoderma koningii, Trichoderma longibrachiatum, Trichoderma reesei, or Trichoderma viride cells.

The polynucleotide encoding the fusion protein is inserted into an “expression vector,” “cloning vector,” or “vector.” Expression vectors can replicate autonomously, or they can replicate by being inserted into the genome of the host cell. Often, it is desirable for a vector to be usable in more than one host cell, e.g., in E. coli for cloning and construction, and in a mammalian cell for expression. Additional elements of the vector can include, for example, selectable markers, e.g., tetracycline resistance or hygromycin resistance, which permit detection and/or selection of those cells transformed with the desired polynucleotide sequences (see, e.g., U.S. Pat. No. 4,704,362). The particular vector used to transport the genetic information into the cell is also not particularly critical. Any suitable vector used for expression of recombinant proteins host cells can be used.

Typically, the polynucleotide that encodes the fusion protein is placed under the control of a promoter that is functional in the desired host cell. An extremely wide variety of promoters are well known, and can be used in the expression vectors of the invention, cell in which the promoter is to be active. Other expression control sequences such as ribosome binding sites, transcription termination sites and the like are also optionally included. Constructs that include one or more of these control sequences are termed “expression cassettes.” Accordingly, the invention provides expression cassettes into which the nucleic acids that encode fusion proteins are incorporated for high level expression in a desired host cell.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) 8: 4057), the tac promoter (DeBoer, et al., Proc. Natl. Acad. Sci. U.S.A. (1983) 80:21-25); and the lambda-derived P_{L} promoter and N-gene ribosome binding site (Shimatake et al., Nature (1981) 292: 128). The particular promoter system is not critical to the invention, any available promoter that functions in prokaryotes can be used.

For expression of fusion proteins in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli.

A ribosome binding site (RBS) is conveniently included in the expression cassettes of the invention. An RBS in E. coli, for example, consists of a nucleotide sequence 3-9 nucleotides in length located 3-11 nucleotides upstream of the initiation codon (Shine and Dalgarno, Nature (1975) 254: 34; Steitz, In Biological regulation and development: Gene expression (ed. R. F. Goldberger), vol. 1, p. 349, 1979, Plenum Publishing, NY).

For expression of the fusion proteins in yeast, convenient promoters include GAL1-10 (Johnson and Davies (1984) Mol. Cell. Biol. 4:1440-1448) ADH2 (Russell et al. (1983) J. Biol. Chem. 258:2674-2682), PHO5 (EMBO J. (1982) 6:675-680), and MFα (Herskowitz and Oshima (1982) in The Molecular Biology of the Yeast Saccharomyces (eds. Strathern, Jones, and Broach) Cold Spring Harbor Lab., Cold Spring Harbor, N.Y., pp. 181-209). Another Cousens et al., Gene 61:265-275 (1987). For filamentous fungi such as, for example, strains of the fungi Aspergillus (McKnight et al., U.S. Pat. No. 4,935,349), examples of useful promoters include those derived from Aspergillus nidulans glycolytic genes, such as the ADH3 promoter (McKnight et al., EMBO J. 4: 2093 2099 (1985)) and the tpiA promoter. An example of a suitable terminator is the ADH3 terminator (McKnight et al.).

Suitable constitutive promoters for use in plants include, for example, the cauliflower mosaic virus (CaMV) 35S transcription initiation region and region VI promoters, the 1′- or 2′-promoter derived from T-DNA of Agrobacterium tumefaciens, and other promoters active in plant cells that are known to those of skill in the art. Other suitable promoters include the full-length transcript promoter from Figwort mosaic virus, actin promoters, histone promoters, tubulin promoters, or the mannopine synthase promoter (MAS). Other constitutive plant promoters include various ubiquitin or polyubiquitin promoters derived from, inter alia, Arabidopsis (Sun and Callis, Plant J., 1 1(5):1017-1027 (1997)), the mas, Mac or DoubleMac promoters (described in U.S. Pat. No. 5,106,739 and by Comai et al., Plant Mol. Biol. 15:373-381 (1990)) and other transcription initiation regions from various plant genes known to those of skill in the art. Useful promoters for plants also include those obtained from Ti- or Ri-plasmids, from plant cells, plant viruses or other hosts where the promoters are found to be functional in plants. Bacterial promoters that function in plants, and thus are suitable for use in the methods of the invention include the octopine synthetase promoter, the nopaline synthase promoter, and the manopine synthetase promoter. Suitable endogenous plant promoters include the ribulose-1,6-biphosphate (RUBP) carboxylase small subunit (ssu) promoter, the (α-conglycinin promoter, the phaseolin promoter, the ADH promoter, and heat-shock promoters.

For mammalian cells, the control sequences will include a promoter and preferably an enhancer derived from immunoglobulin genes, SV40, cytomegalovirus, etc., and a polyadenylation sequence, and may include splice donor and acceptor sequences.

In a preferred embodiment, the fusion proteins of the present invention are expressed in a filamentous fungal host cell, for example, Aspergillus niger. Examples of suitable promoters for expressing the fusion proteins of the present invention in a filamentous fungal host cell are promoters obtained from the genes for Aspergillus oryzae TAKA amylase, Rhizomucor miehei aspartic proteinase, Aspergillus niger neutral α-amylase, glucoamylase (glaA), Rhizomucor miehei lipase, Aspergillus oryzae alkaline protease, Aspergillus oryzae triose phosphate isomerase, Aspergillus nidulans acetamidase, Fusarium oxysporum trypsin-like protease (WO 96/00787), as well as the NA2-tpi promoter (a hybrid of the promoters from the genes for Aspergillus niger neutral α-amylase and Aspergillus oryzae triose phosphate isomerase); and mutant, truncated, and hybrid promoters thereof.

Either constitutive or regulated promoters can be used in the present invention. Regulated promoters can be advantageous because the host cells can be grown to high densities before expression of the fusion proteins is induced. High level expression of heterologous proteins slows cell growth in some situations. An inducible promoter is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, which allow one to control the timing of expression of the glycosyltransferase or enzyme involved in nucleotide sugar synthesis. For E. coli and other bacterial host cells, inducible promoters are known to those of skill in the art. These include, for example, the lac promoter, the bacteriophage lambda P_{L} promoter, the hybrid trp-lac promoter (Amann et al. (1983) Gene 25: 167; de Boer et al. (1983) Proc. Nat'l. Acad. Sci. USA 80: 21), and the bacteriophage T7 promoter (Studier et al. (1986) J. Mol. Biol.; Tabor et al. (1985) Proc. Nat'l. Acad. Sci. USA 82: 1074-8). These promoters and their use are discussed in Sambrook et al., supra. A particularly preferred inducible promoter for expression in prokaryotes is a dual promoter that includes a tac promoter component linked to a promoter component obtained from a gene or genes that encode enzymes involved in galactose metabolism (e.g., a promoter from a UDPgalactose 4-epimerase gene (galE)). The dual tac-gal promoter, which is described in PCT Patent Application Publ. No. WO98/20111, provides a level of expression that is greater than that provided by either promoter alone.

Inducible promoters for use in plants are known to those of skill in the art (see, e.g., references cited in Kuhlemeier et al (1987) Ann. Rev. Plant Physiol. 38:221), and include those of the 1,5-ribulose bisphosphate carboxylase small subunit genes of Arabidopsis thaliana (the “ssu” promoter), which are light-inducible and active only in photosynthetic tissue. [symbol] art. These include, for example, the arabinose promoter, the lacZ promoter, the metallothionein promoter, and the heat shock promoter, as well as many others.

A construct that includes a polynucleotide of interest operably linked to gene expression control signals that, when placed in an appropriate host cell, drive expression of the polynucleotide is termed an “expression cassette.” Expression cassettes that encode the fusion proteins of the invention are often placed in expression vectors for introduction into the host cell. The vectors typically include, in addition to an expression cassette, a nucleic acid sequence that enables the vector to replicate independently in one or more selected host cells. Generally, this sequence is one that enables the vector to replicate independently of the host chromosomal DNA, and includes origins of replication or autonomously replicating sequences. Such sequences are well known for a variety of bacteria. For instance, the origin of replication from the plasmid pBR322 is suitable for most Gram-negative bacteria. Alternatively, the vector can replicate by becoming integrated into the host cell genomic complement and being replicated as the cell undergoes DNA replication. A preferred expression vector for expression of the enzymes is in bacterial cells is pTGK, which includes a dual tac-gal promoter and is described in PCT Patent Application Publ. NO. WO98/20111.

Preferred expression vectors for expression of the fusion proteins of the invention in filamentous fungal host cells, for example, Aspergillus niger, are described in, for example, U.S. Pat. No. 5,364,770, EPO Publication No. 0215594, WO 90/15860. See also, U.S. Pat. Nos. 6,265,204; 6,130,063; 6,103,490; 6,103,464; 6,004,785; 5,679,543; and 5,364,770. Preferred terminators for expression in filamentous fungal host cells are obtained from the genes for Aspergillus oryzae TAKA amylase, Aspergillus niger glucoamylase, Aspergillus nidulans anthranilate synthase, Aspergillus niger α-glucosidase, and Fusarium oxysporum trypsin-like protease. Preferred polyadenylation sequences for expression in filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger α-glucosidase. Effective signal peptide coding regions for expression in filamentous fungal host cells are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humnicola lanuginosa lipase. [symbol] expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the TAKA α-amylase promoter, Aspergillus niger glucoamylase promoter, and Aspergillus oryzae glucoamylase promoter may be used as regulatory sequences. Other examples of regulatory sequences are those that allow for gene amplification. In eukaryotic systems, these include the dihydrofolate reductase gene that is amplified in the presence of methotrexate, and the metallothionein genes which are amplified with heavy metals. In these cases, the nucleic acid sequence encoding the polypeptide would be operably linked with the regulatory sequence.

The construction of polynucleotide constructs generally requires the use of vectors able to replicate in bacteria. A plethora of kits are commercially available for the purification of plasmids from bacteria (see, for example, EasyPrepJ, FlexiPrepJ, both from Pharmacia Biotech; StrataCleanJ, from Stratagene; and, QIAexpress Expression System, Qiagen). The isolated and purified plasmids can then be further manipulated to produce other plasmids, and used to transfect cells. Cloning in Streptomyces or Bacillus is also possible.

Selectable markers are often incorporated into the expression vectors used to express the polynucleotides of the invention. These genes can encode a gene product, such as a protein, necessary for the survival or growth of transformed host cells grown in a selective culture medium. Host cells not transformed with the vector containing the selection gene will not survive in the culture medium. Typical selection genes encode proteins that confer resistance to antibiotics or other toxins, such as ampicillin, neomycin, kanamycin, chloramphenicol, or tetracycline. Alternatively, selectable markers may encode proteins that complement auxotrophic deficiencies or supply critical nutrients not available from complex media, e.g., the gene encoding D-alanine racemase for Bacilli. Often, the vector will have one selectable marker that is functional in, e.g., E. coli, or other cells in which the vector is replicated prior to being introduced into the host cell. A number of selectable markers are known to those of skill in the art and are described for instance in Sambrook et al., supra. A preferred selectable marker for use in bacterial cells is a kanamycin resistance marker (Vieira and Messing, Gene 19: 259 (1982)). Use of kanamycin selection is advantageous over, for example, ampicillin selection because ampicillin is quickly degraded by β-lactamase in [symbol] overgrown with cells that do not contain the vector.

Suitable selectable markers for use in mammalian cells include, for example, the dihydrofolate reductase gene (DHFR), the thymidine kinase gene (TK), or prokaryotic genes conferring drug resistance, gpt (xanthine-guanine phosphoribosyltransferase, which can be selected for with mycophenolic acid; neo (neomycin phosphotransferase), which can be selected for with G418, hygromycin, or puromycin; and DHFR (dihydrofolate reductase), which can be selected for with methotrexate (Mulligan & Berg (1981) Proc. Nat'l. Acad. Sci. USA 78: 2072; Southern & Berg (1982) J. Mol. Appl. Genet. 1: 327).

Selection markers for plant and/or other eukaryotic cells often confer resistance to a biocide or an antibiotic, such as, for example, kanamycin, G 418, bleomycin, hygromycin, or chloramphenicol, or herbicide resistance, such as resistance to chlorsulfuron or Basta. Examples of suitable coding sequences for selectable markers are: the neo gene which codes for the enzyme neomycin phosphotransferase which confers resistance to the antibiotic kanamycin (Beck et al (1982) Gene 19:327); the hyg gene, which codes for the enzyme hygromycin phosphotransferase and confers resistance to the antibiotic hygromycin (Gritz and Davies (1983) Gene 25:179); and the bar gene (EP 242236) that codes for phosphinothricin acetyl transferase which confers resistance to the herbicidal compounds phosphinothricin and bialaphos.

Selectable markers for use in a filamentous fungal host cell include, but are not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

Construction of suitable vectors containing one or more of the above listed components employs standard ligation techniques as described in the references cited above. Isolated plasmids or DNA fragments are cleaved, tailored, and re-ligated in the form desired to generate the plasmids required. To confirm correct sequences in plasmids constructed, the plasmids can be analyzed by standard techniques such as by restriction endonuclease digestion, and/or sequencing according to known methods. Molecular cloning techniques to methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques, Methods in Enzymology, Volume 152, Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1998 Supplement) (Ausubel).

A variety of common vectors suitable fo r use as starting materials for constructing the expression vectors of the invention are well known in the art. For cloning in bacteria, common vectors include pBR322 derived vectors such as pBLUESCRIPT™, and λ-phage derived vectors. In yeast, vectors include Yeast Integrating plasmids (e.g., YIp5) and Yeast Replicating plasmids (the YRp series plasmids) and pGPD-2. Expression in mammalian cells can be achieved using a variety of commonly available plasmids, including pSV2, pBC12BI, and p91023, as well as lytic virus vectors (e.g., vaccinia virus, adeno virus, and baculovirus), episomal virus vectors (e.g., bovine papillomavirus), and retroviral vectors (e.g., murine retroviruses).

The methods for introducing the expression vectors into a chosen host cell are not particularly critical, and such methods are known to those of skill in the art. For example, the expression vectors can be introduced into prokaryotic cells, including E. coli, by calcium chloride transformation, and into eukaryotic cells by calcium phosphate treatment or electroporation. Other transformation methods are also suitable.

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156 and WO 96/00787.

Translational coupling may be used to enhance expression. The strategy uses a short upstream open reading frame derived from a highly expressed gene native to the translational system, which is placed downstream of the promoter, and a ribosome binding site followed after a few amino acid codons by a termination codon. Just prior to the start codon for the initiation of translation. The system dissolves secondary structure in the RNA, allowing for the efficient initiation of translation. See Squires, et. al. (1988), J. Biol. Chem. 263: 16297-16302.

The fusion proteins can be expressed intracellularly, or can be secreted from the cell. Intracellular expression often results in high yields. If necessary, the amount of soluble, active fusion protein may be increased by performing refolding procedures (see, e.g., Sambrook et al., supra.; Marston et al., Bio/Technology (1984) 2: 800; Schoner et al., Bio/Technology (1985) 3:151). In embodiments in which the fusion proteins are secreted from the cell, either into the periplasm or into the extracellular medium, the DNA sequence is linked to a cleavable signal peptide sequence. The signal sequence directs translocation of the fusion protein through the cell membrane. An example of a suitable vector for use in E. coli that contains a promoter-signal sequence unit is pTA1529, which has the E. coli phoA promoter and signal sequence (see, e.g., Sambrook et al., supra.; Oka et al., Proc. Natl. Acad. Sci. USA (1985) 82: 7212; Talmadge et al., Proc. Natl. Acad. Sci. USA (1980) 77: 3988; Takahara et al., J. Biol. Chem. (1985) 260: 2670). In another embodiment, the fusion proteins are fused to a subsequence of protein A or bovine serum albumin (BSA), for example, to facilitate purification, secretion, or stability.

The fusion proteins of the invention can also be further linked to other bacterial proteins. This approach often results in high yields, because normal prokaryotic control sequences direct transcription and translation. In E. coli, lacZ fusions are often used to express heterologous proteins. Suitable vectors are readily available, such as the pUR, pEX, and pMR100 series (see, e.g., Sambrook et al., supra.). For certain applications, it may be desirable to cleave the non-glycosyltransferase and/or accessory enzyme amino acids from the fusion protein after purification. This can be accomplished by any of several methods known in the art, including cleavage by cyanogen bromide, a protease, or by Factor Xₐ (see, e.g., Sambrook et al., supra.; Itakura et al., Science (1977) 198: 1056; Goeddel et al., Proc. Natl. Acad. Sci. USA (1979) 76: 106; Nagai et al., Nature (1984) 309: 810; Sung et al., Proc. Natl. Acad. Sci. USA (1986) 83: 561). Cleavage sites can be engineered into the gene for the fusion protein at the desired point of cleavage. [symbol] multiple transcriptional cassettes in a single expression vector, or by utilizing different selectable markers for each of the expression vectors employed in the cloning strategy.

A suitable system for obtaining recombinant proteins from E. coli which maintains the integrity of their N-termini has been described by Miller et al. Biotechnology 7:698-704 (1989). In this system, the gene of interest is produced as a C-terminal fusion to the first 76 residues of the yeast ubiquitin gene containing a peptidase cleavage site. Cleavage at the junction of the two moieties results in production of a protein having an intact authentic N-terminal reside.

The expression vectors of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Fusion proteins that comprise sequences from eukaryotic glycosyltransferases, may be expressed in, for example, eukaryotic cells, but expression of such proteins are not limited to eukaryotic cells, as described above. In a preferred embodiment, recombinant fucosyltransferase fusion proteins of the present invention are produced in Aspergillus niger cells. Fusion proteins that comprise sequences from prokaryotic glycotransferases may be expressed in, for example, prokaryotic cells, but expression of such proteins are not limited to prokaryotic cells, as described above. For example, a eukaryotic fusion protein may be expressed in a prokaryotic host cell (see, e.g., Fang et al. (1998) J. Am. Chem. Soc. 120: 6635-6638), or vice versa. When fusion proteins are expressed in mammalian cells, the fusion proteins can be a secreted form or can be a membrane bound form that is retained by the cells.

The vectors can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes. One of skill in the art will appreciate that vectors comprising DNA encoding the fusion protein of the invention can conveniently be transfected into different host cells. [symbol] procedures of the art, including ammonium sulfate precipitation, affinity columns, column chromatography, and the like (see, generally, Scopes, PROTEIN PURUFICATION (1982)). Substantially pure compositions of at least about 90 to 95% homogeneity are preferred, and those of 98 to 99% or more homogeneity are most preferred for pharmaceutical uses. Once purified, partially or to homogeneity as desired, the polypeptides may then be used therapeutically and diagnostically.

Methods for refolding single chain polypeptides are described, are well-known and are applicable to the fusion proteins of the invention. (See, e.g., Buchner et al., Analytical Biochemistry 205: 263-270 (1992); Pluckthun, Biotechnology 9: 545 (1991); Huse et al., Science 246: 1275 (1989) and Ward et al., Nature 341: 544 (1989)).

Often, functional protein from E. coli or other bacteria is generated from inclusion bodies and requires the solubilization of the protein using strong denaturants, and subsequent refolding. In the solubilization step, a reducing agent is generally present to disrupt disulfide bonds as is well-known in the art. Renaturation to an appropriate folded form is typically accomplished by dilution (e.g. 100-fold) of the denatured and reduced protein into refolding buffer.

Modification and Domain Swapping

In one embodiment of the present invention the domains of recombinantly produced polypeptides are modified and/or swapped to generate recombinant fusion proteins with a desired level of expression in cells or enzymatic activity (e.g., acceptor substrate specificity or catalytic activity), or starch-binding domain. One of skill will recognize the many ways of manipulating the nucleic acids encoding a polypeptide, or a subsequence thereof, to modify or swap a domain of a polypeptide to generate the fusion proteins of the present invention. Well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Giliman and Smith (1979) Gene 8:81-97, Roberts et al. (1987) Nature 328: 731-734.

For example, a nucleic acid encoding a polypeptide, or a subsequence thereof, can be modified to facilitate the linkage of two functional domains to obtain the polynucleotides can be placed at either end of a domain so that the domain can be linked to the starch-binding domain by, for example, a sulfide linkage. The modification can be done using either recombinant or chemical methods (see, e.g., Pierce Chemical Co. catalog, Rockford Ill.).

The nucleic acids encoding subsequences of a polypeptide, such as a catalytic domain or stem region, can be joined by linker domains, which are typically protein sequences, such as poly-glycine sequences of between about 5 and 200 amino acids, with between about 10-100 amino acids being typical. Proline residues can be incorporated into the linker to prevent the formation of significant secondary structural elements by the linker. Preferred linkers are often flexible amino acid subsequences that are synthesized as part of a recombinant fusion protein. The flexible linker can be an amino acid subsequence comprising a proline such as Gly(x)-Pro-Gly(x) where x is a number between about 3 and about 100. Also, a chemical linker can be used to connect synthetically or recombinantly produced domains of one or more polypeptide. Such flexible linkers are known to persons of skill in the art. For example, poly(ethylene glycol) linkers are available from Shearwater Polymers, Inc. Huntsville, Ala. These linkers can optionally have amide linkages, sulfhydryl linkages, or heterofinctional linkages.

Other useful mutations include, for example, deletions from, or insertions or substitutions of, residues within the amino acid sequence of the polypeptide of interest so that it contains the proper epitope and is able to form a covalent bond with a reactive metal chelate. Any combination of deletion, insertion, and substitution is made to arrive at the final construct, provided that the final construct possesses the desired characteristics. The amino acid changes also may alter post-translational processes of the polypeptide of interest, such as changing the number or position of glycosylation sites.

For the design of amino acid sequence mutants of a polypeptide, the location of the mutation site and the nature of the mutation will be determined by the specific polypeptide of interest being modified. The sites for mutation can be modified individually or in series, e.g., by: (1) substituting first with conservative amino acid choices and then with more radical selections depending upon the results achieved; (2) deleting the target residue; or (3) inserting residues of the same or a different class adjacent to the located site, or combinations of options 1-3. of interest that are preferred locations for mutagenesis is called “alanine scanning mutagenesis,” as described by Cunningham and Wells, Science, 244: 1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with the surrounding aqueous environment in or outside the cell. Those domains demonstrating functional sensitivity to the substitutions then are refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis is conducted at the target codon or region and the variants produced are screened for increased reactivity with a particular reactive chelate.

Amino acid sequence deletions generally range from about 1 to 30 residues, more preferably about 1 to 10 residues, and typically they are contiguous. Contiguous deletions ordinarily are made in even numbers of residues, but single or odd numbers of deletions are within the scope hereof. As an example, deletions may be introduced into regions of low homology among related polypeptides, which share the most sequence identity to the amino acid sequence of the polypeptide of interest to modify the half-life of the polypeptide. Deletions from the polypeptide of interest in areas of substantial homology with one of the binding sites of other ligands will be more likely to modify the biological activity of the polypeptide of interest more significantly. The number of consecutive deletions will be selected so as to preserve the tertiary structure of the polypeptide of interest in the affected domain, e.g., beta-pleated sheet or alpha helix.

Amino acid sequence insertions include amino- and/or carboxyl-terminal fusions ranging in length from one residue to polypeptides containing a hundred or more residues, as well as intra-sequence insertions of single or multiple amino acid residues. Intra-sequence insertions (i.e., insertions within the mature polypeptide sequence) may range generally from about 1 to 10 residues, more preferably 1 to 5, most preferably 1 to 3. Insertions are preferably made in even numbers of residues, but this is not required. Examples of insertions include insertions to the internal portion of the polypeptide of interest, as well as N- or C-terminal fusions with proteins or peptides containing the desired epitope that will result, upon fusion, in an increased reactivity with the chelate. [symbol] at least one amino acid residue in the polypeptide molecule removed and a different residue inserted in its place. Sites of interest for amino acid variation are those in which particular residues of the polypeptide obtained from various species are identical among all animal species of the polypeptide of interest, this degree of conservation suggesting importance in achieving biological activity common to these molecules. These sites, especially those falling within a sequence of at least three other identically conserved sites, are substituted in a relatively conservative manner. Such conservative substitutions are shown in Table 1 under the heading of preferred substitutions. If such substitutions result in a change in biological activity, then more substantial changes, denominated exemplary substitutions in Table 1, or as further described below in reference to amino acid classes, are introduced and the products screened.

TABLE 1

| Original | Substitution |
|---|---|
| Ala (A) | val; leu; ile |
| Arg (R) | lys; gln; asn |
| Asn (N) | gln; his; lys |
| Asp (D) | glu |
| Cys (C) | ser |
| Gln (Q) | asn |
| Glu (E) | asp |
| Gly (G) | pro; ala |
| His (H) | asn; gln; lys; arg |
| Ile (I) | leu; vat; met; ala |
| phe; | norleucine |
| Leu (L) | norleucine; ile; val; met; |
| | ala; phe |
| Lys (K) | arg; gln; asn |
| Met (M) | leu; phe; ile |
| Phe (F) | leu; val; ile; ala; leu |
| Pro (P) | ala |
| Ser (S) | thr |
| Thr (T) | ser |
| Trp (W) | tyr; phe |
| Tyr (Y) | trp; phe; thr; ser |
| Val (V) | ile; leu; met; phe; ala; |
| | norleucine |

Moreover, modifications in the function of the polypeptide of interest can be made by selecting substitutions that differ significantly in their effect on maintaining: (a) the [symbol] or helical conformation; (b) the charge or hydrophobicity of the molecule at the target site; or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions entail exchanging a member of one of the above classes for another class. Such substituted residues also may be introduced into the conservative substitution sites or, more preferably, into the remaining (non-conserved) sites.

It also may be desirable to inactivate one or more protease cleavage sites that are present in the molecule. These sites are identified by inspection of the encoded amino acid sequence, in the case of trypsin, e.g., for an arginyl or lysinyl residue. When protease cleavage sites are identified, they are rendered inactive to proteolytic cleavage by substituting the targeted residue with another residue, preferably a residue such as glutamine or a hydrophilic residue such as serine; by deleting the residue; or by inserting a prolyl residue immediately after the residue.

In another embodiment, any methionyl residues other than the starting methionyl residue of the signal sequence, or any residue located within about three residues N- or C-terminal to each such methionyl residue, is substituted by another residue (preferably in accord with Table 1) or deleted. Alternatively, about 1-3 residues are inserted adjacent to such sites.

The nucleic acid molecules encoding amino acid sequence mutations of the polypeptides of interest are prepared by a variety of methods known in the art. These methods include, but are not limited to, preparation by oligonucleotide-mediated (or site-directed) mutagenesis, PCR mutagenesis, and cassette mutagenesis of an earlier prepared variant or a non-variant version of the polypeptide on which the variant herein is based. [symbol] substitution, deletion, and insertion recognition moiety mutants herein. This technique is well known in the art as described by Ito et al., Gene 102: 67-70 (1991) and Adelman et al., DNA 2: 183 (1983). Briefly, the DNA is altered by hybridizing an oligonucleotide encoding the desired mutation to a DNA template, where the template is the single-stranded form of a plasmid or bacteriophage containing the unaltered or native DNA sequence of the polypeptide to be varied. After hybridization, a DNA polymerase is used to synthesize an entire second complementary strand of the template that will thus incorporate the oligonucleotide primer, and will code for the selected alteration in the DNA.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al., Proc. Natl. Acad. Sci. USA, 75: 5765 (1978).

One preferred method for obtaining specific nucleic acid sequences combines the use of synthetic oligonucleotide primers with polymerase extension or ligation on a mRNA or DNA template. Such a method, e.g., RT, PCR, or LCR, amplifies the desired nucleotide sequence, which is often known (see, U.S. Pat. Nos. 4,683,195 and 4,683,202). Restriction endonuclease sites can be incorporated into the primers. Amplified polynucleotides are purified and ligated into an appropriate vector. Alterations in the natural gene sequence can be introduced by techniques such as in vitro mutagenesis and PCR using primers that have been designed to incorporate appropriate mutations.

Oligonucleotides that are not commercially available are preferably chemically synthesized according to the solid phase phosphoramidite triester method first described by Beaucage & Caruthers, Tetrahedron Letts. 22: 1859-1862 (1981), using an automated synthesizer, as described in Van Devanter et. al., Nucleic Acids Res. 12: 6159-6168 (1984). Purification of oligonucleotides is accomplished by any art-recognized method, e.g., native acrylamide gel electrophoresis or by anion-exchange HPLC as described in Pearson & Reanier, J. Chrom. 255: 137-149 (1983).

If the DNA sequence is synthesized chemically, a single stranded oligonucleotide will result. This may be converted into double stranded DNA by hybridization with a strand as a template. While it is possible to chemically synthesize an entire single chain Fv region, it is preferable to synthesize a number of shorter sequences (about 100 to 150 bases) that are later ligated together.

Alternatively, subsequences may be cloned and the appropriate subsequences cleaved using appropriate restriction enzymes. The fragments may then be ligated to produce the desired DNA sequence.

Nucleic acids encoding SBDs or subsequences thereof are typically cloned into intermediate vectors before transformation into prokaryotic or eukaryotic cells for replication and/or expression. These intermediate vectors are typically prokaryote vectors, e.g., plasmids, or shuttle vectors. Isolated nucleic acids encoding therapeutic proteins comprise a nucleic acid sequence encoding a therapeutic protein and subsequences, interspecies homologues, alleles and polymorphic variants thereof.

The invention is exemplified by reference to the preparation of fusion proteins of glycosyltransferases. Those of skill will recognize that the invention is broadly applicable, not to just glycosyltransferases, but to other enzyme types as well. Additional, non-limiting, representative classes of enzymes of use in the present invention are discussed below.

The Recognition Moiety

The recognition moiety is the species that is immobilized on a support and which is recognized by the SBD with which it interacts immobilizing the composition that includes the SBD on the support. The present invention can be practiced with any recognition moiety that recognizes and interacts with the starch-binding domain. In an exemplary embodiment, the recognition moiety is a saccharide or a species that includes a saccharide.

A presently preferred recognition moiety is a cyclodextrin or modified cyclodextrin. Cyclodextrins are a group of cyclic oligosaccharides produced by numerous microorganisms. Cyclodextrins have a ring structure that has a basket-like shape. This shape allows cyclodextrins to include many kinds of molecules into their internal cavity. See, for example, Szejtli, J., CYCLODEXTRINS AND THEIR INCLUSION COMPLEXES; Akademiai Klado, Budapest, 1982; and Bender et al., CYCLODEXTRIN CHEMISTRY, Springer-Verlag, Berlin, 1978. Cyclodextrins are able to form inclusion complexes with an array of organic molecules including, for example, drugs, pesticides, herbicides and agents of war. See, Tenjarla et al., J. [symbol] Albers et al., Crit. Rev. Ther. Drug Carder Syst. 12:311-337 (1995). Importantly, cyclodextrins are able to discriminate between enantiomers of compounds in their inclusion complexes. See, Koppenhoefer et al. J. Chromatogr. A 793:153-164 (1998).

Preparation of the Solid-Support

The compositions of the invention that include a starch-binding domain are optionally immobilized on a solid support by an interaction between the starch-binding domain and a recognition moiety that is immobilized on a solid support. The recognition moiety is a species that recognizes and interacts with the starch-binding domain. The recognition moiety and the solid support are linked by a bond formed by the reaction between a reactive functional group on the solid support and a reactive functional group of complementary reactivity on the recognition moiety.

Useful reactive functional groups include, for example:
(a) carboxyl groups and various derivatives thereof including, but not limited to, N-hydroxysuccinimide esters, N-hydroxybenztriazole esters, acid halides (e.g., I, Br, Cl), acyl imidazoles, thioesters, p-nitrophenyl esters, alkyl, alkenyl, alkynyl and aromatic esters;
(b) hydroxyl groups, which can be converted to, e.g., esters, ethers, aldehydes, etc.
(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the functional group of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be, for example, converted to disulfides or reacted with acyl halides; [symbol] oxidized;
(i) alkenes, which can undergo, for example, cycloadditions, acylation, Michael addition, etc;
(j) epoxides, which can react with, for example, amines and hydroxyl compounds; and
(k) phosphoramidites and other standard functional groups useful in nucleic acid synthesis.

The reactive functional groups can be chosen such that they do not participate in, or interfere with, the reactions necessary to assemble the recognition moiety or the support. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

In an exemplary embodiment, the recognition moiety is a cyclodextrin. Cyclodextrin polymers have been produced by linking or cross-linking cyclodextrins or mixtures of cyclodextrins and other carbohydrates with polymerizing agents, e.g. epichlorhydrin, diizocynanates, diepoxides (Insoluble cyclodextrin polymer beads, Chem. Abstr. No. 222444m, 102: 94; Zsadon and Fenyvesi, 1st. Int. Symp. on Cyclodextrins, J. Szejtli, ed., D. Reidel Publishing Co., Boston, pp. 327-336; Fenyvesi et al., 1979, Ann. Univ. Budapest, Section Chim. 15: 13 22; and Wiedenhofet al., 1969, Die Stirke 21: 119-123). These polymerizing agents are capable of reacting with the primary and secondary hydroxy groups on carbons 6, 2, and 3. Polymerization will not eliminate the central cavity of cyclodextrin molecules. Stable water soluble cyclodextrin polymers may be formed by linking two to five cyclodextrin units. (Fenyvesi et al. 1st Int. Symp. on Cyclodextrins, J. Szejtli, ed., D. Reider Publishing Co., Boston, p. 345).

Insoluble cyclodextrin polymers can be prepared in the form of beads, fiber, resin or film by cross-linking a large number of cyclodextrin monomers as described in the previous paragraph, supra. Such polymers have the ability to swell in water. The characteristics of the polymeric product, chemical composition, swelling and particle size distribution may be controlled by varying the conditions of preparation. These cyclodextrin polymers have been compounds and aliphatic amino acids from one another (Harada et al., 1982, Chem. Abstr. No. 218351u, 96: 10 and Zsadon and Fenyvesi, 1982, 1st. Int. Symp. on Cyclodextrins, J. Szejtli, ed., D. Reidel Publishing Co., Boston, pp. 327-336). Additionally, beta-cyclodextrin immobilized with epichlorohydrin has been used as a catalyst for the selective synthesis of 4-hydroxybenzaldehyde (Komiyama and Hirai, 1986, Polymer J. 18: 375-377).

Methods of preparing immobilized cyclodextrins are also known in the art. Immobilized cyclodextrins may be obtained using a variety of procedures. One method involves linking vinyl derivatives of cyclodextrin monomers. For example, water soluble polymers containing cyclodextrin have been obtained using acrylic ester derivatives (Harada et al., 1976, J. Am. Chem. Soc. 9: 701-704).

Immobilized cyclodextrins have also been obtained by covalently linking cyclodextrin to a solid surface via a linker arm, or by incorporating them into synthetic polymer matrices by physical methods (Zsadon and Fenyvesi (1982) Ist. Int. Symp. on Cyclodextrins, J. Szejtli, ed., D. Reidel Publishing Co., Boston, pp. 327-336). Cyclodextrin monomers have been attached to silica gel through silanes (Armstrong et al. (1987) Science 232: 1132 and Armstrong U.S. Pat. No. 4,539,399) and by reacting carboxylated silica with ethylenediamine monosubstituted cyclodextrin (Kawaguchi et al. (1983) Anal. Chem., 55: 1852-1857). Cyclodextrin has also been covalently linked to polyurethane resins (Kawaguchi et al. (1982) Bull. Chem. Soc. Jpn. 55: 2611-2614), Sepharose™, BioGel™, cellulose (Zsadon and Fenyvesi (1982) 1st Int. Symp. on Cyclodextrins, J. Szejtli, ed., D. Reidel Publishing Co., Boston, pp. 327-336). Such cyclodextrin containing solid surfaces have been used as stationary phases in the chromatographic separation of aromatic compounds (Kawaguchi et al. (1983) Anal. Chem., 55: 1852-1857). Additionally cyclodextrin has been linked to polyacrylamide (Tanaka (1982) J. Chromatog. 246: 207-214 and Tanaka et al. (1981) Anal. Let. 14:281-290).

Both charged an uncharged cyclodextrins and derivatives of cyclodextrins are known in the art. In a preferred embodiment, the recognition moiety is an uncharged cyclodextrin.

The cyclodextrin affinity moiety can also be attached to the support via a spacer arm. See, Yamamoto et al., J. Phys. Chem. B 101: 6855-6860 (1997). Methods to attach [symbol] pharmaceutical arts. See, Sreenivasan, K. J. Appl. Polym. Sci. 60: 2245-2249 (1996).

An exemplary strategy involves incorporation of a protected sulfhydryl onto the recognition moiety using the heterobifunctional crosslinker SPDP (n-succinimidyl-3-(2-pyridyldithio)propionate and then deprotecting the sulfhydryl for formation of a disulfide bond with another sulfhydryl on the solid support. In the protected form, the SPDP generated sulfhydryl on the recognition moiety react with the free sulfhydryls incorporated onto the solid support forming a disulfide bond. SPDP reacts with primary amines and the incorporated sulfhydryl is protected by 2-pyridylthione.

As those of skill in the art will appreciate, many other crosslinkers are of use in preparing the solid support of the present invention. Examples include 2-iminothiolane or N-succinimidyl S-acetylthioacetate (SATA), available for forming disulfide bonds. 2-iminothiolane reacts with primary amines, instantly incorporating an unprotected sulfhydryl onto the protein. SATA also reacts with primary amines, but incorporates a protected sulfhydryl, which is later deacetaylated using hydroxylamine to produce a free sulfhydryl. In each case, the incorporated sulfhydryl is free to react with other sulfhydryls or protected sulfhydryl, like SPDP, forming the required disulfide bond.

The above-described strategies are exemplary and not limiting of linkers of use in the invention. Other crosslinkers are available that can be used. For example, TPCH(S-(2-thiopyridyl)-L-cysteine hydrazide and TPMPH ((S-(2-thiopyridyl) mercapto-propionohydrazide) react with carbohydrate moieties that have been previously oxidized by mild periodate treatment, thus forming a hydrazone bond between the hydrazide portion of the crosslinker and the periodate generated aldehydes. The modification is site-specific and will not interfere with the ability of the recognition moiety to bind to the starch-binding domain. TPCH and TPMPH introduce a 2-pyridylthione protected sulfhydryl group onto the recognition moiety, which can be deprotected with DTT and then subsequently used for conjugation, such as forming disulfide bonds between components.

If disulfide bonding is found unsuitable for producing stable conjugates, other crosslinkers may be used that incorporate more stable bonds between components. The heterobifunctional crosslinkers GMBS (N-gamma-maleimidobutyryloxy)succinirnide) and SMCC (succinimidyl 4-(N-maleimido-methyl)cyclohexane) react with primary amines, thus introducing a maleimide group onto the component. This maleimide group can subsequently [symbol] mentioned crosslinkers, thus forming a stable thioether bond between the components. If steric hindrance between components interferes with either component's activity, crosslinkers can be used which introduce long spacer arms between components and include derivatives of some of the previously mentioned crosslinkers (i.e., SPDP). Thus there is an abundance of suitable crosslinkers, which are useful; each of which is selected depending on the effects it has on optimal immunoconjugate production.

A variety of reagents are of use to bind the recognition moiety to the solid phase. See, Wold, F., Meth. Enzymol. 25: 623-651, 1972; Weetall, H. H., and Cooney, D. A., In: ENZYMES AS DRUGS. (J. S. Holcenberg, and J. Roberts, eds.) pp. 395-442, Wiley, New York, 1981; Ji, T. H., Meth. Enzymol. 91: 580-609, 1983; Mattson et al., Mol. Biol. Rep. 17: 167-183, 1993, all of which are incorporated herein by reference). Useful crosslinking reagents are derived from various zero-length, homo-bifunctional, and hetero-bifunctional crosslinking reagents. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. In addition to these chemical reagents, the enzyme transglutaminase (glutamyl-peptide γ-glutamyltransferase; EC 2.3.2.13) may be used as zero-length crosslinking reagent. This enzyme catalyzes acyl transfer reactions at carboxamide groups of protein-bound glutaminyl residues, usually with a primary amino group as substrate. Preferred homo- and hetero-bifunctional reagents contain two identical or two dissimilar sites, respectively, which may be reactive for amino, sulfhydryl, guanidino, indole, or nonspecific groups.

Preferred Specific Sites in Crosslinking Reagents

1. Amino-Reactive Groups

In one preferred embodiment, the linker arm is formed from a reagent that includes an amino-reactive group. Useful non-limiting examples of amino-reactive groups include N-hydroxysuccinimide (NHS) esters, imidoesters, isocyanates, acylhalides, arylazides, p-nitrophenyl esters, aldehydes, and sulfonyl chlorides. [symbol] of the affinity component. The imidazole groups of histidines are known to compete with primary amines for reaction, but the reaction products are unstable and readily hydrolyzed. The reaction involves the nucleophilic attack of an amine on the acid carboxyl of an NHS ester to form an amide, releasing the N-hydroxysuccinimide. Thus, the positive charge of the original amino group is lost.

Imidoesters are the most specific acylating reagents for reaction with amine groups. At a pH between 7 and 10, imidoesters react only with primary amines. Primary amines attack imidates nucleophilically to produce an intermediate that breaks down to amidine at high pH or to a new imidate at low pH. The new imidate can react with another primary amine, thus crosslinking two amino groups, a case of a putatively monofunctional imidate reacting bifunctionally. The principal product of reaction with primary amines is an amidine that is a stronger base than the original amine. The positive charge of the original amino group is therefore retained. As a result, imidoesters do not affect the overall charge of the conjugate.

Isocyanates (and isothiocyanates) react with to form stable bonds. Their reactions with sulfhydryl, imidazole, and tyrosyl groups give relatively unstable products.

Acylazides are also used as amino-specific reagents in which nucleophilic amines of the affinity component attack acidic carboxyl groups under slightly alkaline conditions, e.g. pH8.5.

Arylhalides such as 1,5-difluoro-2,4-dinitrobenzene react preferentially with the amino groups and tyrosine phenolic groups of the conjugate components, but also with its sulfhydryl and imidazole groups.

p-Nitrophenyl esters of mono- and dicarboxylic acids are also useful amino-reactive groups. Although the reagent specificity is not very high, α- and ε-amino groups appear to react most rapidly.

Aldehydes such as glutaraldehyde react with primary amines (e.g., ε-amino group of lysine residues). Glutaraldehyde, however, displays reactivity with several other amino acid side chains including those of cysteine, histidine, and tyrosine. Since dilute glutaraldehyde solutions contain monomeric and a large number of polymeric forms (cyclic hemiacetal) of glutaraldehyde, the distance between two crosslinked groups within the affinity component with the aldehydes of the polymer, glutaraldehyde is capable of modifying the affinity component with stable crosslinks. At pH 6-8, the pH of typical crosslinking conditions, the cyclic polymers undergo a dehydration to form α-β unsaturated aldehyde polymers. Schiff bases, however, are stable, when conjugated to another double bond. The resonant interaction of both double bonds prevents hydrolysis of the Schiff linkage. Furthermore, amines at high local concentrations can attack the ethylenic double bond to form a stable Michael addition product.

Aromatic sulfonyl chlorides react with a variety of sites, but reaction with the amino groups is the most important, resulting in a stable sulfonamide linkage.

2. Sulfhydryl-Reactive Groups

In another preferred embodiment, the linker arm is formed from a reagent that includes a sulfhydryl-reactive group. Useful non-limiting examples of sulfhydryl-reactive groups include maleimides, alkyl halides, pyridyl disulfides, and thiophthalimides.

Maleimides react preferentially with sulfhydryl groups to form stable thioether bonds. They also react at a much slower rate with primary amino groups and the imidazole groups of histidines. However, at pH 7 the maleimide group can be considered a sulfhydryl-specific group, since at this pH the reaction rate of simple thiols is 1000-fold greater than that of the corresponding amine.

Alkyl halides react with sulfhydryl groups, sulfides, imidazoles, and amino groups. At neutral to slightly alkaline pH, however, alkyl halides react primarily with sulfhydryl groups to form stable thioether bonds. At higher pH, reaction with amino groups is favored.

Pyridyl disulfides react with free sulfhydryls via disulfide exchange to give mixed disulfides. As a result, pyridyl disulfides are the most specific sulfhydryl-reactive groups.

Thiophthalimides react with free sulfhydryl groups to form also disulfides.

3. Guanidino-Reactive Groups

In another embodiment, the linker arm is formed from a reagent that includes a guanidino-reactive group. A useful non-limiting example of a guanidino-reactive group is phenylglyoxal. Phenylglyoxal reacts primarily with the guanidino groups of arginine [symbol] extent.

4. Indole-Reactive Groups

In another embodiment, the sites are indole-reactive groups. Useful non-limiting examples of indole-reactive groups are sulfenyl halides. Sulfenyl halides react with tryptophan and cysteine, producing a thioester and a disulfide, respectively. To a minor extent, methionine may undergo oxidation in the presence of sulfenyl chloride.

5. Carboxyl-Reactive Residue

In another embodiment, carbodiimides soluble in both water and organic solvent, are used as carboxyl-reactive reagents. These compounds react with free carboxyl groups forming a pseudourea that can then couple to available amines yielding an amide linkage (Yamada et al., Biochemistry 20: 4836-4842, 1981) teach how to modify a protein with carbodiimde.

Preferred Nonspecific Sites in Crosslinking Reagents

In addition to the use of site-specific reactive moieties, the present invention contemplates the use of non-specific reactive groups to link the mutant recognition moiety to the solid support. Non-specific groups include photoactivatable groups, for example. In another preferred embodiment, the sites are photoactivatable groups. Photoactivatable groups, completely inert in the dark, are converted to reactive species upon absorption of a photon of appropriate energy. In one preferred embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently preferred. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, [symbol] photolysis conditions for the affinity component than unsubstituted arylazides.

In another preferred embodiment, photoactivatable groups are selected from fluorinated arylazides. The photolysis products of fluorinated arylazides are arylnitrenes, all of which undergo the characteristic reactions of this group, including C—H bond insertion, with high efficiency (Keana et al., J. Org. Chem. 55: 3640-3647, 1990).

In another embodiment, photoactivatable groups are selected from benzophenone residues. Benzophenone reagents generally give higher crosslinking yields than arylazide reagents.

In another embodiment, photoactivatable groups are selected from diazo compounds, which form an electron-deficient carbene upon photolysis. These carbenes undergo a variety of reactions including insertion into C—H bonds, addition to double bonds (including aromatic systems), hydrogen attraction and coordination to nucleophilic centers to give carbon ions.

In still another embodiment, photoactivatable groups are selected from diazopyruvates. For example, the p-nitrophenyl ester of p-nitrophenyl diazopyruvate reacts with aliphatic amines to give diazopyruvic acid amides that undergo ultraviolet photolysis to form aldehydes. The photolyzed diazopyruvate-modified affinity component will react like formaldehyde or glutaraldehyde forming intraprotein crosslinks.

Homobifunctional Reagents

1. Homobifunctional Crosslinkers Reactive with Primary Amines

Synthesis, properties, and applications of homobifunctional amine-reactive reagents are described in the literature (for reviews of crosslinking procedures and reagents, see above). Many reagents are available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional NHS esters include disuccinimidyl glutarate (DSG), disuccinimidyl suberate (DSS), bis(sulfosuccinimidyl) suberate (BS), disuccinimidyl tartarate (DST), disulfosuccinimidyl tartarate (sulfo-DST), bis-2-(succinimidooxycarbonyloxy)ethylsulfone (BSOCOES), bis-2-(sulfosuccinimidooxy-carbonyloxy)ethylsulfone (sulfo-BSOCOES), ethylene glycolbis(succinimidylsuccinate) (EGS), ethylene glycolbis(sulfosuccinimidylsuccinate) (sulfo-EGS), dithiobis(succinimidyl- [symbol] limiting examples of homobifunctional imidoesters include dimethyl malonimidate (DMM), dimethyl succinimidate (DMSC), dimethyl adipimidate (DMA), dimethyl pimelimidate (DMP), dimethyl suberimidate (DMS), dimethyl-3,3′-oxydipropionimidate (DODP), dimethyl-3,3′-(methylenedioxy)dipropionimidate (DMDP), dimethyl-,3′-(dimethylenedioxy)dipropionimidate (DDDP), dimethyl-3,3′-(tetramethylenedioxy)-dipropionimidate (DTDP), and dimethyl-3,3′-dithiobispropionimidate (DTBP).

Preferred, non-limiting examples of homobifunctional isothiocyanates include: p-phenylenediisothiocyanate (DITC), and 4,4′-diisothiocyano-2,2′-disulfonic acid stilbene (DIDS).

Preferred, non-limiting examples of Homobifunctional isocyanates include xylene-diisocyanate, toluene-2,4-diisocyanate, toluene-2-isocyanate-4-isothiocyanate, 3-methoxydiphenylmethane-4,4′-diisocyanate, 2,2′-dicarboxy-4,4′-azophenyldiisocyanate, and hexamethylenediisocyanate.

Preferred, non-limiting examples of Homobifunctional arylhalides include 1,5-difluoro-2,4-dinitrobenzene (DFDNB), and 4,4′-difluoro-3,3′-dinitrophenyl-sulfone.

Preferred, non-limiting examples of Homobifunctional aliphatic aldehyde reagents include glyoxal, malondialdehyde, and glutaraldehyde.

Preferred, non-limiting examples of homobifunctional acylating reagents include nitrophenyl esters of dicarboxylic acids.

Preferred, non-limiting examples of Homobifunctional aromatic sulfonyl chlorides include phenol-2,4-disulfonyl chloride, and .alpha.-naphthol-2,4-disulfonyl chloride.

Preferred, non-limiting examples of additional amino-reactive homobifunctional reagents include erythritolbiscarbonate which reacts with amines to give biscarbamates.

2. Homobifunctional Crosslinkers Reactive with Free Sulfhydryl Groups

Synthesis, properties, and applications of sulfhydryl-reactive reagents are described in the literature (for reviews of crosslinking procedures and reagents, see above). Many of the reagents are commercially available (e.g., Pierce Chemical Company, Rockford, Ill.; Sigma Chemical Company, St. Louis, Mo.; Molecular Probes, Inc., Eugene, Oreg.). [symbol]
bismaleimidohexane (BMH), N,N′-(1,3-phenylene) bismaleimide, N,N′-(1,2-phenylene)bismaleimide, azophenyldimaleimide, and bis(N-maleimidomethyl)ether.
Preferred, non-limiting examples of homobifunctional pyridyl disulfides include 1,4-di->3′-(2′-pyridyldithio)propionamidobutane (DPDPB).

Preferred, non-limiting examples of homobifunctional alkyl halides include 2,2′-dicarboxy-4,4′-diiodoacetamidoazobenzene, α,α′-diiodo-p-xylenesulfonic acid, α,α′-dibromo-p-xylenesulfonic acid, N,N′-bis(b-bromoethyl)benzylamine, N,N′-di(bromoacetyl)phenylthydrazine, and 1,2-di(bromoacetyl)amino-3-phenylpropane.

3. Homobifunctional Photoactivatable Crosslinkers

Synthesis, properties, and applications of photoactivatable reagents are described in the literature (for reviews of crosslinking procedures and reagents, see above). Some of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of Homobifunctional photoactivatable crosslinker include bis-b-(4-azidosalicylamido)ethyldisulfide (BASED), di-N-(2-;nitro-4-azidophenyl)-cystamine-S,S-dioxide (DNCO), and 4,4′-dithiobisphenylazide.

Hetero-Bifunctional Reagents

1. Amino-Reactive Hetero-Bifunctional Reagents with a Pyridyl Disulfide Moiety

Synthesis, properties, and applications of heterobifunctional sulfhydryl-reactive reagents are described in the literature (for reviews of crosslinking procedures and reagents, see above). Many of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of hetero-bifunctional reagents with a pyridyl disulfide moiety and an amino-reactive NHS ester include N-succinimidyl-3-(2-pyridyldithio)propionate (SPDP), succinimidyl 6-3-(2-pyridyldithio)propionamidohexanoate (LC-SPDP), sulfosuccinimidyl 6-3-(2-pyridyldithio)propionamidohexanoate (sulfo-LCSPDP), 4-succinimidyloxycarbonyl-a-methyl-α-(2-pyridyldithio)toluene (SMPT), and sulfosuccinimidyl 6-a-methyl-α-(2-pyridyldithio)toluamidohexanoate (sulfo-LC-SMPT).

Synthesis, properties, and applications of heterobifunctional amine/sulfhydryl-reactive reagents are described in the literature. Preferred, non-limiting examples of hetero-bifunctional reagents with a maleimide moiety and an amino-reactive NHS ester include succinimidyl maleimidylacetate (AMAS), succinimidyl 3-maleimidylpropionate (WMPS), N-γ-maleimidobutyryloxysuccinimide ester (GMBS)N-γ-maleimidobutyryloxysulfo succinimide ester (sulfo-GMBS) succinimidyl 6-maleimidylhexanoate (EMCS), succinimidyl 3-maleimidylbenzoate (SMB), m-maleimidobenzoyl-N-hydroxysuccinimide ester (MBS), m-maleimidobenzoyl-N-hydroxysulfosuccinimide ester (sulfo-MBS), succinimidyl 4-(N-maleimidomethyl)-cyclohexane-1-carboxylate (SMCC), sulfosuccinimidyl 4-(N-maleimidomethyl)cyclohexane-1-carboxylate (sulfo-SMCC), succinimidyl 4-(p-maleimidophenyl)butyrate (SMPB), and sulfosuccinimidyl 4-(p-maleimidophenyl)butyrate (sulfo-SMPB).

3. Amino-Reactive Hetero-Bifunctional Reagents with an Alkyl Halide Moiety

Preferred, non-limiting examples of hetero-bifunctional reagents with an alkyl halide moiety and an amino-reactive NHS ester include N-succinimidyl-(4-iodoacetyl)aminobenzoate (SIAB), sulfosuccinimidyl-(4-iodoacetyl)aminobenzoate (sulfo-SIAB), succinimidyl-6-(iodoacetyl)aminohexanoate (SIAX), succinimidyl-6-(6-((iodoacetyl)-amino)hexanoylamino)hexanoate (SIAXX), succinimidyl-6-(((4-(iodoacetyl)-amino)-methyl)-cyclohexane-1-carbonyl)aminohexanoate (SIACX), and succinimidyl-4((iodoacetyl)-amino)methylcyclohexane-1-carboxylate (SIAC).

A preferred example of a hetero-bifunctional reagent with an amino-reactive NHS ester and an alkyl dihalide moiety is N-hydroxysuccinimidyl 2,3-dibromopropionate (SDBP). SDBP introduces intramolecular crosslinks to the affinity component by conjugating its amino groups. The reactivity of the dibromopropionyl moiety for primary amino groups is controlled by the reaction temperature (McKenzie et al., Protein Chem. 7: 581-592 (1988)).

Preferred, non-limiting examples of hetero-bifunctional reagents with an allyl halide moiety and an amino-reactive p-nitrophenyl ester moiety include p-nitrophenyl iodoacetate (NPIA).

NEWS Ester Moiety

Preferred, non-limiting examples of photoactivatable arylazide-containing hetero-bifunctional reagents with an amino-reactive NHS ester include N-hydroxysuccinimidyl4-azidosalicylic acid (NHS-ASA), N-hydroxysulfosuccinimidyl-4-azidosalicylic acid (sulfo-NHS-ASA), sulfosuccinimidyl-(4-azidosalicylamido)hexanoate (sulfo-NHS-LC-ASA), N-hydroxysuccinimidyl N-(4-azidosalicyl)-6-aminocaproic acid (NHS-ASC), N-hydroxy-succinimidyl-4-azidobenzoate (HSAB), N-hydroxysulfo-succinimidyl-4-azidobenzoate (sulfo-HSAB), sulfosuccinimidyl-4-(p-azidophenyl)butyrate (sulfo-SAPB), N-5-azido-2-nitrobenzoyloxy-succinimide (ANB-NOS), N-succinimidyl-6-(4′-azido-2′-nitrophenyl-amino)hexanoate (SANPAH), sulfosuccinimidyl-6-(4′-azido-2′-nitrophenylamino)-hexanoate (sulfo-SANPAH), N-succinimidyl 2-(4-azidophenyl)dithioacetic acid (NHS-APDA), N-succinimidyl-(4-azidophenyl)1,3′-dithiopropionate (SADP), sulfosuccinimidyl-(4-azidophenyl)-1,3′-dithiopropionate (sulfo-SADP), sulfosuccinimidyl-2-(m-azido-o-nitrobenzamido)ethyl-1,3′-dithiopropionate (SAND), sulfosuccinimidyl-2-(p-azidosalicylamido)-ethyl-1,3′-dithiopropionate (SASD), N-hydroxysuccinimidyl 4-azidobenzoylglycyltyrosine (NHS-ABGT), sulfosuccinimidyl-2-(7-azido-4-4-methylcoumarin-3-acetamide)ethyl-1,3′-dithiopropionate (SAED), and sulfosuccinimidyl-7-azido-4-methylcoumarin-3-acetate (sulfo-SAMCA).

Other cross-linking agents are known to those of skill in the art (see, for example, Pomato et al., U.S. Pat. No. 5,965,106.

Linker Groups

In addition to the embodiments set forth above, wherein the cross-linking moiety is attached directly to a site on the recognition moiety and on the support, the present invention also provides constructs in which the cross-linking moiety is bound to a site present on a linker group that is bound to either the recognition moiety or the solid support or both.

In certain embodiments, it is advantageous to tether the recognition moiety to the solid support by a group that provides flexibility and increases the distance between the mutant recognition moiety and the targeting moiety. Properties that are usefully controlled include, for example, hydrophobicity, hydrophilicity, surface-activity and the distance of the recognition moiety from the chromatographic support. [symbol] from the chromatographic support. Linkers with this characteristic have several uses. For example, a recognition moiety held too closely to the support may not effectively interact with the SBD, or it may interact with too low of an affinity. Thus, it is within the scope of the present invention to utilize linker moieties to, inter alia, vary the distance between the recognition moiety and the chromatographic support.

In yet a further embodiment, the linker group is provided with a group that can be cleaved to release the recognition moiety from the support. Many cleavable groups are known in the art. See, for example, Jung et al., Biochem. Biophys. Acta, 761: 152-162 (1983); Joshi et al., J. Biol. Chem., 265: 14518-14525 (1990); Zarling et al., J. Immunol., 124: 913-920 (1980); Bouizar et al., Eur. J. Biochem., 155: 141-147 (1986); Park et al., J. Biol. Chem., 261: 205-210 (1986); Browning et al., J. Immunol., 143: 1859-1867 (1989). Moreover, a broad range of cleavable, bifunctional (both homo- and hetero-bifunctional) linker groups are commercially available from suppliers such as Pierce.

Exemplary cleavable moieties are cleaved using light, heat or reagents such as thiols, hydroxylamine, bases, periodate and the like. Exemplary cleavable groups comprise a cleavable moiety which is a member selected from the group consisting of disulfide, ester, imide, carbonate, nitrobenzyl, phenacyl and benzoin groups.

The Kit

In yet another aspect, the invention provides a kit for practicing a method of the invention. The kit contains one or more of the components described herein and, typically, instructions for using the component(s). In an exemplary embodiment, the kit includes a saccharide-modified solid support and one or more enzyme that includes a SBD. In yet another exemplary embodiment, the enzyme is a glycosyltransferase or other enzyme that transfers a glycosyl donor to a substrate.

The following examples are offered to illustrate, but not to limit the claimed invention.

### EXAMPLE 1

Procedure to make Beta Cyclodextrin Affinity Resin [symbol] place in an open chromatography resin.

2. Hydrate the resin with 100 mL DI water, allowing resin to drain in column at room temperature.

3. Remove resin from column and place in a 50 mL Falcon tube.

4. Dissolve 11 g of beta cyclodextrin (BCD, Sigma Cat # C-4767) in 20 mL of 1M NaOH. (˜0.4 M solution of BCD)

5. Add BCD solution to resin in 50 mL tube. Final volume=47 mL.

6. Place resulting suspension in 40-45° C. water bath for 48-72 hours.

7. Pour resin into chromatography column and rinse with 100 mL DI water. Allow to drain.

8. Remove resin from column and place in a clean 50 mL Falcon tube. Add 1M ethanolamine to resin to a total suspension volume of 50 mL and incubate 17-24 hours at 40° C.

9. Rinse resin in column with 100 mL DI water, and allow to drain.

10. Place resin into 50 mL Falcon tube, and add 0.1M NaOH to a total volume of 40 mL.

11. Store resin in 0.1M NaOH at room temperature in capped tube.

12 To use resin for chromatography, pack column of desired volume. Equilibrate with appropriate buffer. Elute bound target protein with 5 mM BCD in equilibration buffer.

### EXAMPLE 2

Starch Binding Domain Construct

The Starch Binding Domain(SBD) gene was isolated by PCR of pGAST ampr. The oligonucleotides used in the PCR are 5′SBDNedI (5′-AGGTATCATATGTGTACCACTCCCACCGCCGT-3′; SEQ ID NO. 6) and 3′SBDBAMH (5′-GTTTATGGATCCCCGCCAGGTGTCGGTCAC-3′; SEQ ID NO. 7). The SBD PCR reaction was analyzed by agarose gel electrophoresis, and the ˜330 bp band was gel purified. The pCWIN2 and gel purified SBD PCR product were digested by NdeI and BamfHI restriction endonucleases, and the reactions were analyzed by agarose gel electrophoresis. The digestion products representing the linear vector (˜5 kb) and SBD (˜330 bp) were then gel purified. The digested gel purified vector and insert were ligated together using T4 DNA [symbol] tranformants were identified by restriction endonuclease screening. A transformant was shown to contain a ˜330 bp insert, and following sequencing it was proven that the insert is the SBD. The pCWIN2SBD was then transformed into chemically competent JM109 E.coli (JMCB006), and a positive transformant was identified by restriction endonuclease screening. A 125 mL culture of the pCWIN2SBD JM109 was induced with 500 μM IPTG and expressed at 25° C. for 17 h. The cells were collected by centrifugation and lysed by French pressing. SDS-PAGE analysis was inconclusive, and a sample from the lysate given to Downstream Processing showed binding to the P-cyclodextran resin, however, the purified product was too large to be the SBD.

Subcloning of ST3GalIII into pCWIN2SBD JM109

The pGEX ST3GalIII DNA and pCWIN2SBD were both digested with BamHI and EcoRI restriction endonucleases, and analyzed by agarose gel electrophoresis. The band fragments representing the linear pWIN2SBD (˜5.3 kb) and ST3GalIII (˜1 kb) were gel purified, and ligated using T4 DNA Ligase. The ligation products were then transformed into electrocompetent DH5αE. coli, and positive transformants were identified by restriction endonuclease screening. A positive transformant was isolated, and was subsequently transformed into salt competent JM109 (JMCB006). A JM109 colony was found to contain the pCWIN2SBDST3GalIII by restriction endonuclease analysis. Two 200 mL cultures were induced, one with 500 μM IPTG and grown at 25° C. for 17 hours, and the second with 1 mM IPTG and grown at 37° C. for 17 h. The cells from these two cultures were collected by centrifugation, and lysed by French pressing. SDS-PAGE and Western blotting using an antibody against ST3GalIII suggested the expression of the SBD-ST3GalIII, however, the majority of the protein was found to be soluble and the similar signal intensities between the uninduced and induced samples may suggest a weak promoter sequence.

It is understood that the examples and embodiments described herein are for illustrative purposes only and that various modifications or changes in light thereof will be suggested to persons skilled in the art and are to included within the spirit and purview of this application and are considered within the scope of the appended claims. All publications, patents, and patent applications cited herein are hereby incorporated by reference in their entirety for all purposes.

1. A method of glycosylating a substrate, said method comprising:
(a) contacting a glycosyl donor moiety and an acceptor for said glycosyl donor moiety with a glycosyltransferase comprising a starch-binding domain under conditions suitable to transfer said glycosyl donor moiety to said substrate; and
(b) immobilizing said glycosyltransferase comprising a starch-binding domain to a solid support comprising a cyclodextrin by binding said starch-binding domain to said cyclodextrin.

2. The method according to claim 1, wherein step (a) is performed prior to step (b).

3. The method according to claim 1, wherein step (b) is performed prior to step (a).

4. The method according to claim 1, wherein said substrate is a member selected from carbohydrates, peptides, glycopeptides, lipids, sphingosines and ceramides.

5. The method according to claim 1, wherein said cyclodextrin is an uncharged cyclodextrin.

6. The method according to claim 1, wherein said cyclodextrin is bound to said solid support through a linker arm.

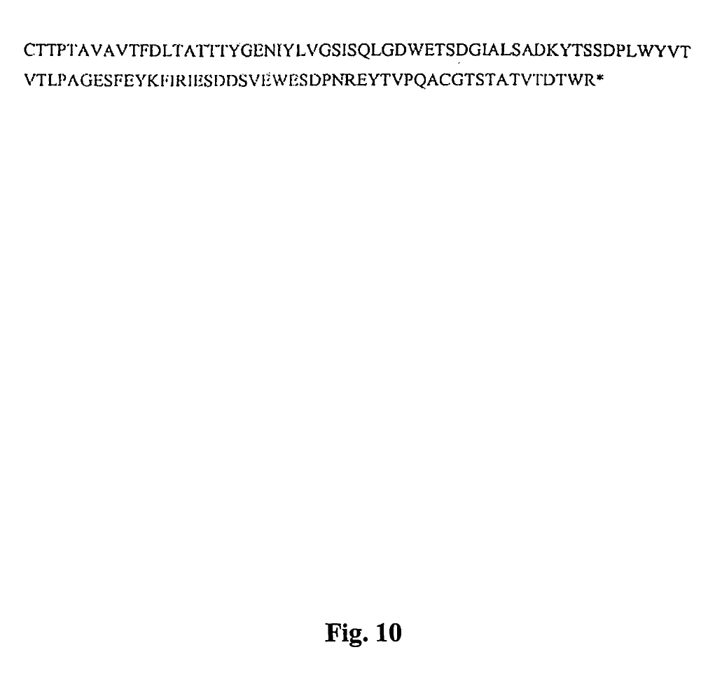

7. The method according to claim 1, wherein said starch-binding domain is a peptide encoded by a nucleic acid comprising the sequence according to FIG. 10.

8. The method according to claim 1, wherein said starch-binding domain is the starch-binding domain of glucoamylase.

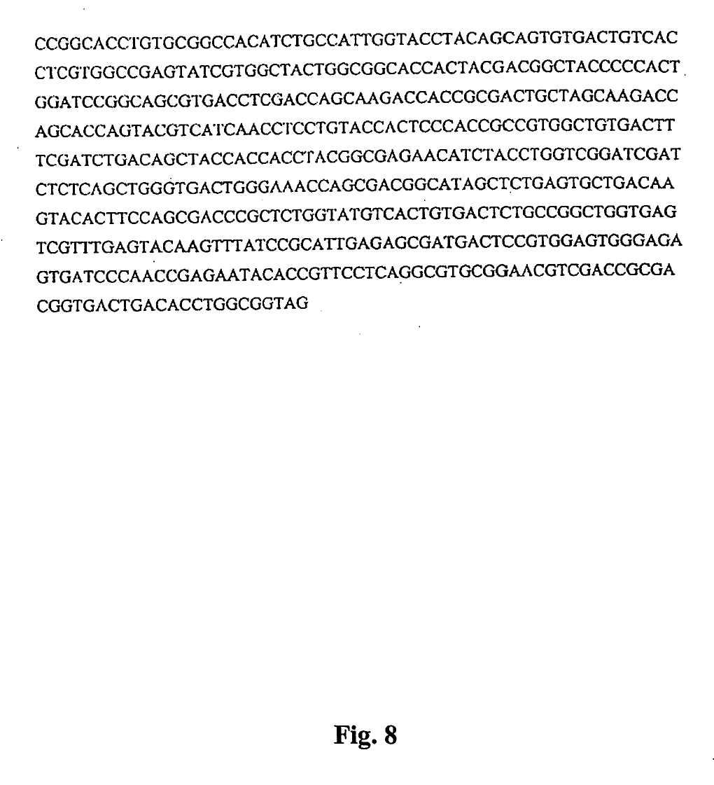

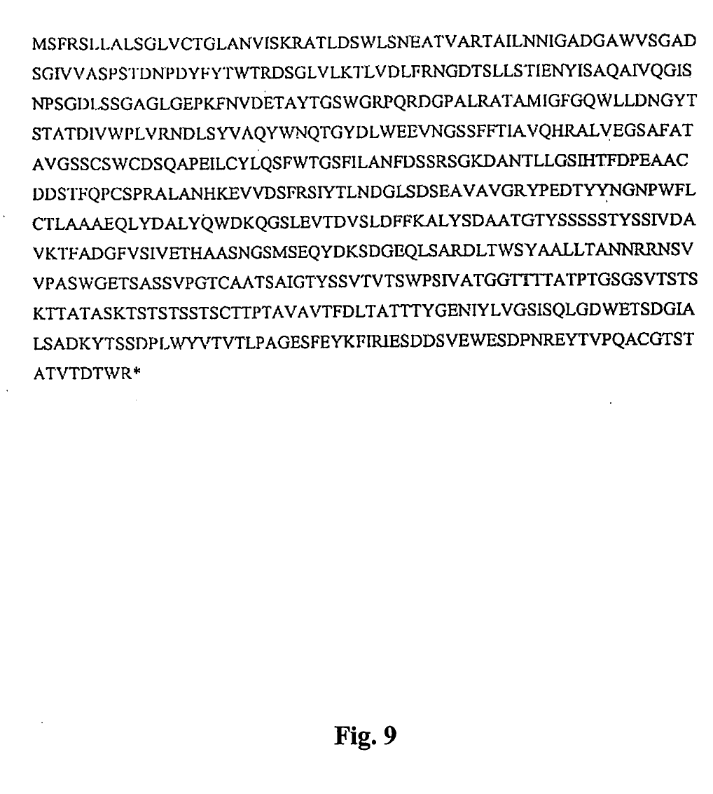

9. The method according to claim 8, wherein said starch-binding domain comprises an amino acid sequence according to FIG. 8.

10. A method of performing an enzymatic transformation on a substrate, said method comprising: [symbol] perform said transformation, wherein said enzyme comprises a starch-binding domain; and
(b) immobilizing said enzyme on a solid support comprising a cyclodextrin by binding said starch-binding domain to said cyclodextrin.

11. The method according to claim 10, wherein step (a) is performed prior to step (b).

12. The method according to claim 10, wherein step (b) is performed prior to step (a).

13. The method according to claim 10, wherein said substrate is a member selected from carbohydrates, peptides, glycopeptides, lipids, sphingosines and ceramides.

14. The method according to claim 8, wherein said cyclodextrin is an uncharged cyclodextrin.

15. The method according to claim 8, wherein said cyclodextrin is bound to said solid support through a linker arm.

16. The method according to claim 10, wherein said starch binding domain is the starch-binding domain of glucoamylase.

17. The method according to claim 7, wherein said starch-binding domain is a peptide encoded by a nucleic acid comprising the sequence according to FIG. 8.

18. The method according to claim 16, wherein said starch-binding domain comprises an amino acid sequence according to FIG. 10.

19. The method according to claim 8, wherein said enzyme is a member selected from glycosyltransferases, amidases, endoglycanases, sulfotransferases, and trans-sialidases.

20. A material comprising:
(a) a solid support having a cyclodextrin moiety bound thereto; and [symbol] said starch-binding moiety interacts with said cyclodextrin immobilizing said enzyme on said solid support.

21. The material according to claim 20, wherein said cyclodextrin is an uncharged cyclodextrin.

22. The material according to claim 20, wherein said cyclodextrin is bound to said solid support through a linker arm.

23. The material according to claim 20, wherein said starch binding domain is the starch-binding domain of glucoamylase.

24. The material according to claim 20, wherein said starch-binding domain is a peptide encoded by a nucleic acid comprising the sequence according to FIG. 8

25. The material according to claim 23, wherein said starch-binding domain comprises an amino acid sequence according to FIG. 10.

26. The material according to claim 20, wherein said enzyme is a member selected from glycosyltransferases, amidases, endoglycanases, sulfotransferases, and trans-sialidases.

27. A material comprising:
(a) a solid support having a cyclodextrin moiety bound thereto; and
(b) a species comprising a starch-binding moiety, said starch-binding moiety interacting with said cyclodextrin immobilizing said species on said solid support.

28. The material according to claim 27, wherein said cyclodextrin is an uncharged cyclodextrin.

29. The material according to claim 27, wherein said cyclodextrin is bound to said solid support through a linker arm.

30. The material according to claim 27, wherein said starch binding domain is the starch-binding domain of glucoamylase.

31. The material according to claim 27, wherein said starch-binding domain is a peptide encoded by a nucleic acid comprising the sequence according to FIG. 8.

32. The material according to claim 27, wherein said starch-binding domain comprises an amino acid sequence according to FIG. 10.

33. The material according to claim 27, wherein said species is a member selected from enzymes, therapeutic agents, diagnostic agents, biomolecules.


SEQUENCE LISTING (7 sequences)

SEQ ID NO: 1 - protein, 8 aa, Artificial sequence
  FLAG tag
DYKDDDDK

SEQ ID NO: 2 - DNA, 1862 bp, A. awamori
  misc_feature: glaA
gaattcaagctagatgctaagcgatattgcatggcaatatgtgttgatgcatgtgcttcttccttcagcttcccctcgtgcagatgaaggtttggctataaattgaagtggttggtcggggttccgtgaggggctgaagtgcttcctcccttttagacgcaactgagagcctgagcttcatccccagcatcattacacctcagcaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggtctcgtcctcaagaccctcgtcgatctcttccgaaatggagataccagtctcctctccaccattgagaactacatctccgcccaggcaattgtccagggtatcagtaacccctctggtgatctgtccagcggcgctggtctcggtgaacccaagttcaatgtcgatgagactgcctacactggttcttggggacggccgcagcgagatggtccggctctgagagcaactgctatgatcggcttcgggcaatggctgcttgtatgttctccacccccttgcgtctgatctgtgacatatgtagctgactggtcaggacaatggctacaccagcaccgcaacggacattgtttggcccctcgttaggaacgacctgtcgtatgtggctcaatactggaaccagacaggatatggtgtgtttgttttattttaaatttccaaagatgcgccagcagagctaacccgcgatcgcagatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggtctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggtaagtctacgctagacaagcgctcatgttgacagagggtgcgtactaacagaagtaggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacctctgccagcagcgtgc

SEQ ID NO: 3 - DNA, 515 bp, A. awamori
  misc_feature: SBD domain
ccggcacctgtgcggccacatctgccattggtacctacagcagtgtgactgtcacctcgtggccgagtatcgtggctactggcggcaccactacgacggctacccccactggatccggcagcgtgacctcgaccagcaagaccaccgcgactgctagcaagaccagcaccagtacgtcatcaacctcctgtaccactcccaccgccgtggctgtgactttcgatctgacagctaccaccacctacggcgagaacatctacctggtcggatcgatctctcagctgggtgactgggaaaccagcgacggcatagctctgagtgctgacaagtacacttccagcgacccgctctggtatgtcactgtgactctgccggctggtgagtcgtttgagtacaagtttatccgcattgagagcgatgactccgtggagtgggagagtgatcccaaccgagaatacaccgttcctcaggcgtgcggaacgtcgaccgcgacggtgactgacacctggcggtag

SEQ ID NO: 4 - protein, 640 aa, A. awamori
  MISC_FEATURE: G1 form of glucoamylase
MSFRSLLALSGLVCTGLANVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 5 - protein, 108 aa, A. awamori [sequence omitted]

SEQ ID NO: 6 - DNA, 32 bp, Artificial sequence
  5′SBDNedI
aggtatcatatgtgtaccactcccaccgccgt

SEQ ID NO: 7 - DNA, 30 bp, Artificial sequence
  3′SBDBAMHI
gtttatggatccccgccaggtgtcggtcac